SEQUENCE LISTING (56 sequences)

SEQ ID NO: 1 - protein, 401 aa, Homo sapiens
MGSSHHHHHHSSGLVPRGSHMASMTGGQQMGRGSEMAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNHHHHHH

SEQ ID NO: 2 - protein, 394 aa, Homo sapiens
MGSSHHHHHHSSGLVPRGSHMDYKDDDDKAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNHHHHHH

SEQ ID NO: 3 - protein, 323 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMHHHHHH

SEQ ID NO: 4 - protein, 476 aa, Homo sapiens
MASMTGGQQMGRGSEFMDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCAHHHHHH

SEQ ID NO: 5 - protein, 507 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFLESRGPFEQKLISEEDLNMHTEHHHHHH

SEQ ID NO: 6 - protein, 485 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFHHHHHH

SEQ ID NO: 7 - DNA, 34 bp, Homo sapiens
gagacttaataacaaagacctgaagcagagtcag

SEQ ID NO: 8 - DNA, 42 bp, Homo sapiens
ctcgagacttaataatttgcataccctgaaggcaggagtcag

SEQ ID NO: 9 - DNA, 38 bp, Homo sapiens
ctcgagacttaatacacgtgacctgaaggcagagtcag

SEQ ID NO: 10 - DNA, 37 bp, Homo sapiens
ctgactctgccttcaggtcaccctattaagtctcgag

SEQ ID NO: 11 - DNA, 20 bp, Homo sapiens
ttcagcagcgtgttcgactt

SEQ ID NO: 12 - DNA, 20 bp, Homo sapiens
aggaatcgctttctgggtca

SEQ ID NO: 13 - DNA, 21 bp, Homo sapiens
ctaactcccccatggagtcgg

SEQ ID NO: 14 - DNA, 19 bp, Homo sapiens
gtcgatggacgtctcgtgc

SEQ ID NO: 15 - DNA, 22 bp, Homo sapiens
gatggattccagttcgagtatg

SEQ ID NO: 16 - DNA, 22 bp, Homo sapiens
gtttgggttgcttgtctgtttg

SEQ ID NO: 17 - DNA, 20 bp, Homo sapiens
gatgacactgccacctctga

SEQ ID NO: 18 - DNA, 20 bp, Homo sapiens
gactggcggggtgtaagtaa

SEQ ID NO: 19 - DNA, 21 bp, Homo sapiens
atgagagccctcacactcctc

SEQ ID NO: 20 - DNA, 19 bp, Homo sapiens
cgtagaagcgccgataggc

SEQ ID NO: 21 - DNA, 20 bp, Homo sapiens
taatgggctcctttcacctg

SEQ ID NO: 22 - DNA, 20 bp, Homo sapiens
cactgggcagacagtcagaa

SEQ ID NO: 23 - DNA, 20 bp, Homo sapiens
tcagcattttgggaatggcc

SEQ ID NO: 24 - DNA, 20 bp, Homo sapiens
gaggttgttgtcttcgaggt

SEQ ID NO: 25 - DNA, 20 bp, Homo sapiens
agtagggcctggatcttctt

SEQ ID NO: 26 - DNA, 20 bp, Homo sapiens
ctgcttctcagtcagaaggt

SEQ ID NO: 27 - DNA, 20 bp, Homo sapiens
agctggcgcacctcaagatg

SEQ ID NO: 28 - DNA, 20 bp, Homo sapiens
agggaagttgggctcaggac

SEQ ID NO: 29 - DNA, 20 bp, Homo sapiens
gaggcggccagttatcagga

SEQ ID NO: 30 - DNA, 20 bp, Homo sapiens
gttctcctcgccctctagca

SEQ ID NO: 31 - DNA, 21 bp, Homo sapiens
catgtacgttgctatccaggc

SEQ ID NO: 32 - DNA, 21 bp, Homo sapiens
ctccttaatgtcacgcacgat

SEQ ID NO: 33 - DNA, 20 bp, Homo sapiens
cccacatgaagcgacttccc

SEQ ID NO: 34 - DNA, 21 bp, Homo sapiens
caggtccaggagatcgttgaa

SEQ ID NO: 35 - DNA, 21 bp, Homo sapiens
tttgtgggcctgaagaaaact

SEQ ID NO: 36 - DNA, 21 bp, Homo sapiens
agggctgtcctgaataagcag

SEQ ID NO: 37 - DNA, 19 bp, Homo sapiens
ggctcctggcaaaaggtca

SEQ ID NO: 38 - DNA, 20 bp, Homo sapiens
ctgcgtagttgtgctgatgt

SEQ ID NO: 39 - DNA, 21 bp, Homo sapiens
gccgagtggaaacttttgtcg

SEQ ID NO: 40 - DNA, 22 bp, Homo sapiens
ggcagcgtgtacttatccttct

SEQ ID NO: 41 - DNA, 20 bp, Homo sapiens
taatgggctcctttcacctg

SEQ ID NO: 42 - DNA, 19 bp, Homo sapiens
cagtgcgtcttgaggagca

SEQ ID NO: 43 - DNA, 21 bp, Homo sapiens
caggtggaggcaaatcttcgt

SEQ ID NO: 44 - DNA, 22 bp, Homo sapiens
accctgttaatccgttcgtttt

SEQ ID NO: 45 - DNA, 19 bp, Homo sapiens
gcagccacggcctattaag

SEQ ID NO: 46 - DNA, 19 bp, Homo sapiens
ccaccacgtacttgccact

SEQ ID NO: 47 - DNA, 21 bp, Homo sapiens
gcagcctttgtgaaccaacac

SEQ ID NO: 48 - DNA, 20 bp, Homo sapiens
ccccgcacactaggtagaga

SEQ ID NO: 49 - DNA, 20 bp, Homo sapiens
atctccccatacgaagtgcc

SEQ ID NO: 50 - DNA, 22 bp, Homo sapiens
cgtgagctttggtggatttcat

SEQ ID NO: 51 - DNA, 19 bp, Homo sapiens
ggactgccacgctttagca

SEQ ID NO: 52 - DNA, 21 bp, Homo sapiens
tgggtctcgtgtgttttctct

SEQ ID NO: 53 - DNA, 19 bp, Homo sapiens
cttcagcccgtacctggag

SEQ ID NO: 54 - DNA, 19 bp, Homo sapiens
ggagaggaagtcgtggtgc

SEQ ID NO: 55 - DNA, 20 bp, Homo sapiens
gggatcagctccgtggatct

SEQ ID NO: 56 - DNA, 23 bp, Homo sapiens
tgcactttggtactcttgaagtt

(19) United States
(12) Patent Application Publication
(10) Pub. No.: US 2016/0304840 A1
(43) Pub. Date: Oct. 20, 2016

(54) Method for Producing Induced Pluripotent Stem Cells

(71) Applicant: NEW ENGLAND BIOLABS, INC., Ipswich, MA (US)

(72) Inventors: Dapeng Sun, Arlington, MA (US); Fanfan Chen, Ipswich, MA (US); Xianghui Li, Shanghai (CN); Ling Yu, Ipswich, MA (US); Yanye Feng, Ipswich, MA (US)

(73) Assignee: New England Biolabs, Inc., Ipswich, MA (US)

(21) Appl. No.: 15/030,185

(22) PCT Filed: Oct. 31, 2014
(86) PCT No.: PCT/US14/63456

§ 371 Date: Apr. 18, 2016

Related U.S. Application Data
(60) Provisional application No. 61/899,075, filed on Nov. 1, 2013.
(60) Provisional application No. 61/987,774, filed on May 2, 2014.
(60) Provisional application No. 61/993,751, filed on May 15, 2014.

(51) Int. Cl. C12N 7/00; C12N 5/0793; C12N 5/077; C12N 5/071; A61K 35/39; A61L 27/38; A61K 35/35; A61K 35/32; A61K 35/30; C12N 5/074; A61K 35/545

(52) U.S. Cl. CPC C12N 7/00; C12N 5/0696; C12N 5/0619; C12N 5/0653; C12N 5/0654; C12N 5/0676; A61K 35/545; A61L 27/3834; A61L 27/3804; A61K 35/35; A61K 35/32; A61L 27/3821; A61K 35/30; A61L 27/383; A61K 35/39; C12N 2760/18823; C12N 2760/18842; C12N 2501/602; C12N 2501/603; C12N 2501/604; C12N 2501/606; C12N 2501/998; C12N 2506/1307; A61L 2430/02; A61L 2430/34; A61L 2430/32

(57) ABSTRACT

Described herein is an inactivated viral particle comprising one or more transcription factor proteins packaged within the particle. A method for using the particle to make induced pluripotent stem cells is also provided.

### BACKGROUND

Induced pluripotent stem cells (iPSCs) are adult cells that have been genetically reprogrammed into an embryonic stem (ES) cell-like state. iPSCs are similar to ES cells in many aspects, such as the expression of stem cell markers, chromatin methylation patterns, doubling time, embryoid body formation, teratoma formation, viable chimera formation, the ability to differentiate into all three germ layers, and the ability to contribute to many different tissues after injection into an embryo. Induced pluripotent cells have been made from adult stomach, liver, skin cells, blood cells, prostate cells and urinary tract cells, as well as many other tissues. Mouse iPSCs were first reported in 2006, and human iPSCs were first reported in late 2007.

iPSCs have been successfully produced by introducing DNA encoding reprogramming transcription factors into somatic cells using by viral vectors (see, e.g., Takahashi, Cell, 126:663-76 (2006)). The efficiency of this method was reported to be extremely low (in the order of 0.01-0.1%). Moreover, there is a risk that the transfected DNA will insert into the genome of the cell, possibly causing deleterious mutations. Also, because many reprogramming factors are oncogenes, there is a higher risk of tumor formation if the transfected DNA inserts into the genome of a cell.

Several investigators have attempted to reprogram somatic cells using isolated proteins (see, e.g., Zhou, Cell Stem Cell, 4:381-384 (2009), Zhang, Biomaterials, 33: 5047-5055 (2012), Khan, Biomaterials, 34:5336-5343 (2013) and Nemes, Tissue Engineering: Part C. 20:383-392 (2014)). However, such methods describe the use of large amounts of folded protein (up to 8 μg/ml, for example) and, where reported, those methods were extremely inefficient (in the range of 0.001%) and slow (see, e.g., Hu, Stem Cells and Development, 23:1285-1300 (2014)). In some cases, these methods required the use of highly toxic compounds, e.g., a hemolytic exotoxin such as streptolysin O, in order to permeabilize the cells. Given these difficulties in the art, there is still a need for efficient ways to re-program somatic cells to become iPSCs.

### SUMMARY

This disclosure provides, among other things, an inactivated viral particle comprising: an envelope; and one or more (e.g., one, two, three, four or more) isolated transcription factor proteins packaged within the particle. The packaged transcription factors may be reprogramming transcription factors (which induce stem cells from differentiated cells), lineage specifying transcription factors (which induce differentiation of a stem cell), and trans-differentiation transcription factors (which cause one somatic cell type (e.g., fibroblasts) to differentiate into another somatic cell type (e.g., neurons or adipocytes)). In certain embodiments, the transcription factors may be selected from the group consisting of Sox2, Oct4, Klf4, c-Myc, Oct4, C/EBPβ, GATA3 and NeuroD1, although other suitable transcription factors involved in cell differentiation/reprogramming (e.g., Nanog, LIN28 also see Table 3 below) are known and in certain cases may be substituted for another transcription factor that is used herein.

As will be explained in greater detail below, the use of inactivated viral particle to deliver isolated transcription factors to somatic cells can increase the rate of reprogramming to up to 5%, 10%, 15%, 20%, 25% or 30% of the initial cell population

In some embodiments, the inactivated viral particle may be an inactivated Sendai virus, herpes virus, parainfluenza virus or lenti virus particle comprising an HVJ envelope and one or more the isolated transcription factor proteins packaged within the particle.

In some embodiments the inactivated viral particle may be a Sendai viral particle comprising an HVJ envelope and one or more of the isolated transcription factor proteins packaged within the particle.

Also provided is a method comprising transfecting somatic cells with the inactivated viral particle, thereby introducing the one or more transcription factor proteins into the somatic cells. In these embodiments, the introduction of the one or more transcription factor causes the somatic cells to develop into a different cell type, i.e., a cell type that is different from the original cell.

In some embodiments, the inactivated viral particle may comprise one or more of Sox2, Oct4, Klf4 and c-Myc and the method causes the somatic cells to develop into pluripotent stem cells. In these embodiments, the particles may comprise Sox2, Oct4, Klf4 and c-Myc.

In other embodiments, the inactivated viral particle may comprise Oct4 and C/EBPβ and the method causes the somatic cells (non-adipocytic cells) to develop into adipocytes.

In other embodiments, the inactivated viral particle may comprise isolated Sox2, GATA3 and NeuroD1 proteins and the method causes the somatic cells to develop into neurons.

In some cases, the Sox2 transcription factor may have an amino acid sequence that is at least 80% identical to a mammalian Sox2 protein, the Oct4 transcription factor may an amino acid sequence that is at least 80% identical to a mammalian Oct4 protein, the Klf4 transcription factor may have an amino acid sequence that is at least 80% identical to a mammalian Klf4 protein, and the c-Myc transcription factor may have an amino acid sequence that is at least 80% identical to a mammalian c-Myc protein.

In some embodiments, the particle may comprise: a) an Oct4 transcription factor, wherein the Oct4 transcription factor has an amino acid sequence that is at least 80%, 85% or 90% identical to a mammalian Oct4 protein; and b) an C/EBPβ transcription factor, wherein the C/EBPβ transcription factor has an amino acid sequence that is at least 80%, 85% or 90% identical to mammalian C/EBPβ protein, packaged within the particle. In other embodiments, the particle may comprise: a) an Sox2 transcription factor, wherein the Sox2 transcription factor has an amino acid sequence that is at least 80%, 85% or 90% identical to a mammalian Sox2 protein; b) an GATA3 transcription factor, wherein the GATA3 transcription factor has an amino acid sequence that is at least 80% identical to mammalian GATA3 protein; and c) a NeuroD1 transcription factor, wherein the NeuroD1 transcription factor has an amino acid sequence that is at least 80% identical to mammalian NeuroD1 protein.

In some embodiments, the transfecting may be done in vivo, i.e., by administering the inactivated viral particle to an animal. In these embodiments, the viral particle may be administered systemically (e.g., to the bloodstream of the animal). However, in some embodiments the viral particle may be administered directly to a tissue of interest.

In other embodiments, the transfecting may done in vitro, i.e., to a cell grown in culture. In these embodiments, the method may comprise, after the cells have been transfected, culturing the somatic cells on a growth medium to produce pluripotent stem cells. In some cases, the somatic cells are fibroblasts, although other cell types may be used. In some cases, the resultant iPSCs may be administered to an animal. In other embodiments, the method may further comprise culturing the iPSCs on a differentiation medium to cause the iPSCs to differentiate into a differentiated cell type. In these embodiments, the differentiated cells can be administered into a recipient subject in need of the differentiated cells.

Also provided is a method of making the inactivated viral particle summarized above. In these embodiments, the method may comprise combining one or more transcription factor proteins selected from the group consisting of Sox2, Oct4, Klf4, c-Myc, Oct4, C/EBPβ, GATA3 and NeuroD1 with HVJ envelope, e.g., in the presence of a detergent. This method may comprise centrifuging the one or more transcription factor proteins, HVJ envelope and detergent to collect an inactivated viral particle comprising the one or more transcription factor proteins packaged therein.

Also provided herein is a screening method. In certain embodiments, the screening method may comprise: a) transfecting somatic cells with an inactivated viral particle as summarized above; b) contacting a test agent with the somatic cells (which can be done before, during or after the transfecting step); c) culturing the somatic cells; and d) determining whether the test agent has any effect on the cell type produced by culturing step c).

In some cases, the culturing step c) may comprise culturing the somatic cells on pluripotent stem cell induction medium, and the determining step d) may comprise determining whether the test agent has any effect on the induction of pluripotent stem cells. In other embodiments, the culturing step c) may comprises culturing the somatic cells on pluripotent stem cell induction medium to produce pluripotent stem cells and optionally culturing the pluripotent stem cells on a differentiation medium; and the determining step d) may comprise determining whether the test agent has any effect on the differentiation of a second type of somatic cells grown on the differentiation medium. In some cases, the second type of somatic cells may be a different cell type relative to the somatic cells that are induced to become pluripotent stem cells.

The test agent used in the screening assay may be of any type, e.g., a small molecule, a nucleic acid (which may or may not encode a protein) or an isolated protein, e.g., a peptide or another transcription factor. In certain cases, the test agent, e.g., a protein, may be packaged within the inactivated viral particle so that it can be transferred into the somatic cell at the same time as the one or more transcription factors.

Also provided is a screening method comprising: a) packaging a test agent within an inactivated viral particle in the absence of transcription factors, for example isolated Sox2, Oct4, Klf4, c-Myc, Oct4, C/EBPβ, GATA3 and NeuroD1 proteins or nucleic acid encoding the same; b) transfecting an induced pluripotent stem cell with the inactivated viral particle of step a); c) culturing the transfected cells on a differentiation medium; and d) determining whether the test agent has any effect on the cell type produced by culturing step.

The compositions and methods summarized above will be described in greater below.

### BRIEF DESCRIPTION OF THE FIGURES

FIG. 1 shows summary of the timeline for fibroblast reprogramming.

FIG. 2 FACS plots that show there is no significant effect of HVJ-E transfection on fibroblasts.

FIG. 3 is composed of four panels and shows the localization of HVJ-E transfected transcription factors. This figure shows that transcription factors could be transfected into the nucleus of fibroblasts by HVJ-E. The proteins could stay in the nuclear at least for 72 hours (3 days).

FIG. 4 shows the results of flow cytometry analysis of the induced iPSCs.

FIG. 5 shows the results of immunofluorescence analysis of the induced iPSCs.

FIG. 6 shows the results of an Alkaline Phosphatase (AKP) Activity Assay.

FIG. 7 shows the results of Q-PCR analysis of induced iPSCs versus fibroblasts.

FIG. 8 shows the results of Q-PCR analysis of induced iPSCs+/−CD24.

FIG. 9A shows the results of in vitro adipogenic differentiation of iPSCs and RT-PCR (FIG. 9B),Q-PCR (FIG. 9C) analysis of mRNA from the differentiated adipocytes versus the undifferentiated fibroblasts.

FIG. 10A-C shows results of in vitro osteogenic differentiation of iPSCs. Osteicalcin (FIG. 10A), Alizarin Red staining (FIG. 10B) and RT-PCR (FIG. 10C) analysis of osteogenic cells.

FIG. 11A-B shows results of in vitro neurogenic differentiation of iPSCs. Nestin, glial fibrillary acidic protein (GFAP), microtubule-associated protein 2 (MAP2) and β-Tubulin III immunofluorescence staining (FIG. 11A) and RT-PCR analysis (FIG. 11B) of ceurogenic cells.

FIG. 12A-B shows in vitro pancreatic islet cell differentiation. (FIG. 12A) Pdx1, Glucagon and insulin positive pancreatic islet cells. Q-PCR analysis (FIG. 12B) comparing pancreatic induced iPSCs and fibroblasts and the results of a glucose stimulated insulin secretion assay (FIG. 12C).

FIG. 13A-F is composed of six panels and shows osteogenic differentiation in vivo. Osteogenic fibroblasts β-Tricalcium phosphate (β-TCP) scaffold (FIG. 13B) was implanted subcutaneously on the back of nude mice for two months. HE (FIG. 13C), Masson Trichrome (FIG. 13D) and Von Kussa Staining (FIG. 13E) all indicate the new bone formation in osteogenic cell scaffold contract, HLA-ABC immunohistochemistry staining (FIG. 13F) confirmed the osteogenic tissue were originated from human cells.

FIG. 14A-F is composed of six panels and shows adipogenic differentiation in vivo where adipogenic differentiation was achieved according to Example 4. Adipogenic fibroblasts polyglycolic acids (PGA) scaffold (FIG. 14A) was implanted subcutaneously on the back of nude mice for two months, iPSCs adhere to scaffold (FIG. 14B). Adipose tissue in vivo is shown in FIG. 14C. The arrow shows the under graded PGA scaffold in vivo (FIG. 14D). The lipid vacuoles were identified using HE staining (FIG. 14E). HLA-ABC immunohistochemistry staining confirmed the osteogenic tissue were originated from human cells (FIG. 14F).

FIG. 15A-B is composed of two panels and shows teratomas formation in vivo. A fibroblast suspension (1×10⁷ cells) that was reprogrammed as described in Example 5 were mixed with matrigel and injected subcutaneously into SCID mice without anesthesia. After two months tissues from endoderm ((1)), mesoderm ((2)) and ectodermal ((3)) were formed in the teratoma, this confirms the pluripotency of reprogrammed fibroblasts. FIG. 15A shows the stained teratoma tissue. FIG. 15B shows the HLA-ABC staining.

FIG. 16A-E shows adipogenic reprogramming in vivo. FIG. 16A shows the pathway of direct re-programming from fibroblasts to adipogenic cells. FIG. 16B shows direct adipogenic reprogramming from fibroblasts; and two weeks after direct adipogenic reprogramming (FIG. 16C). The fibroblasts change to round pre-adipocyte. Two weeks after induction in adipogenic differention medium, lipid droplets appeared in the cells (FIG. 16D). q-PCR analysis confirmed that CCAAT Enhancer Binding Protein α (C/EBPα) and peroxisome proliferator activated receptor-γ (PPARγ) gene expression increased in directly adipogenic reprogramming and adipogenic differentiation groups (FIG. 16E).

FIG. 17A-F shows direct neurogenic reprogramming. After been directly reprogrammed for 4 times, fibroblasts were induced to neuron cells by directly reprogramming (FIG. 17A). Reprogramming occurred with Nestin (FIG. 17B), GFAP (FIG. 17C), MAP2 (FIG. 17D) and β-Tubullin III (FIG. 17F). The expression level of Nesin and MAP2 increased significantly after directly neurogenic.

FIG. 18 shows results of a further FACS analysis.

FIG. 19 shows the results of a further AKP activity assay.

FIG. 20A-B shows that cells transduced with combinations of the four transcription factors can differentiate into adipocytes. FIG. 20A shows the results from Oil Red O staining. FIG. 20B shows the results from RT-PCR of Adipogenic differentiation.

FIG. 21 shows that RT-PCR analysis indicates that cells transduced with combinations of four transcription factors can differentiate into osteoblasts (Osteocalcin, Cbfa1, Osterix, Collagen I and Osteonectin).

### DEFINITIONS

Before the present invention is further described, it is to be understood that this invention is not limited to particular embodiments described, as such may of course vary. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this invention belongs. All references cited herein are incorporated in full by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Throughout this application, various publications, patents and published patent applications are cited. The disclosures of these publications, patents and published patent applications referenced in this application are hereby incorporated by reference in their entirety into the present disclosure. Citation herein by Applicant of a publication, patent, or published patent application is not an admission by Applicant of the publication, patent, or published patent application as prior art.

It must be noted that as used herein and in the appended claims, the singular forms “a”, “and”, and “the” include plural referents unless the context clearly dictates otherwise. Thus, for example, reference to “a polypeptide” includes a plurality of such polypeptides, and reference to “the compound” includes reference to one or more compounds and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The terms “polypeptide” and “protein” are used interchangeably throughout the application and mean at least two covalently attached amino acids, which includes proteins, polypeptides, oligopeptides and peptides. A polypeptide may be made up of naturally occurring amino acids and peptide bonds, synthetic peptidomimetic structures, or a mixture thereof. Thus “amino acid”, or “peptide residue”, as used herein encompasses both naturally occurring and synthetic amino acids and includes optical isomers of naturally occurring (genetically encodable) amino acids, as well as analogs thereof.

In general, polypeptides may be of any length, e.g., greater than 2 amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids. “Peptides” are generally greater than 2 amino acids, greater than 4 amino acids, greater than about 10 amino acids, greater than about 20 amino acids, usually up to about 10, 20, 30, 40 or 50 amino acids. In certain embodiments, peptides are between 3 and 5 or 5 and 30 amino acids in length. In certain embodiments, a peptide may be three or four amino acids in length.

The term “fusion protein” or grammatical equivalents thereof is meant a protein composed of a plurality of polypeptide components that while typically unjoined in their native state, typically are joined by their respective amino and carboxyl termini through a peptide linkage to form a single continuous polypeptide. Fusion proteins may be a combination of two, three or even four or more different proteins. The term polypeptide includes fusion proteins, including, but not limited to, a fusion of two or more heterologous amino acid sequences, a fusion of a polypeptide with: a heterologous targeting sequence, a linker, an immunologically reactive tag, a purification sequence, or a detectable fusion partner, such as a fluorescent protein, β-galactosidase, luciferase, etc., and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular and may contain modifications in the backbone to increase stability and half-life of such molecules in physiological environments. The nucleic acid may be double stranded, single stranded, or contain portions of both double stranded or single stranded sequence. As will be appreciated by those in the art, the depiction of a single strand (“Watson”) also defines the sequence of the other strand (“Crick”).

The term “endogenous”, when used in reference to a biopolymer, means that which is naturally produced (e.g., by an unmodified mammalian or human cell). As used herein, the terms “endogenous”, “native” and “wild-type” are interchangeable.

The term “assessing” includes any form of measurement, and includes determining if an element is present or not. The terms “determining”, “measuring”, “evaluating”, “assessing” and “assaying” are used interchangeably and includes quantitative and qualitative determinations. Assessing may be relative or absolute. “Assessing the presence of” includes determining the amount of something present, and/or determining whether it is present or absent.

As used herein the term “isolated,” when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “isolated”, with respect to a cell, refers to a cell that is cultured, or otherwise obtained in vitro. If a mammal is described as containing isolated cells, then those isolated cells were obtained in vitro and then implanted into the animal.

As used herein, the term “substantially pure” refers to a compound that is removed from its natural environment and is at least 60% free, preferably at least 75% free, and most preferably at least 90% free from other components with which it is naturally associated. The term “purified” means that the recited material comprises at least about 75% by weight of the total protein, with at least about 80% being preferred, and at least about 90% being particularly preferred.

“Subject,” “individual,” “host” and “patient” are used interchangeably herein, to refer to an animal, human or non-human, that may be susceptible to or have a disorder amenable to therapy according to the methods described herein. Generally, the subject is a mammalian subject. Exemplary subjects include, but are not necessarily limited to, humans, non-human primates, mice, rats, cattle, sheep, goats, pigs, dogs, cats, and horses, with humans being of particular interest.

As used herein, the term “induced pluripotent stem cell” refers to differentiated somatic cells that have been genetically reprogrammed to have stem cell features, e.g., the ability to differentiate into three germ layers (the endoderm, mesoderm, and ectoderm) and can produce germline chimera when they are transplanted into a blastocyst. Induced pluripotent stem cell have been reviewed in several hundred publications, including Robinton, et al., Nature, 481: 295-305 (2012), Mostoslaysky, et al., Stem Cells, 30:28-32 (2012) and Okita, et al., Philos. Trans. R. Soc. Lond. B. Biol. Sci., 366: 2198-2207 (2011). Tests for identifying pluripotent stem cells are well known and some of these methods are described in, e.g., WO 2007/69666, Ichisaka, Nature, 448: 313-317 (2007) and Moad, European Urology, 64: 753-761 (2013). The term “induced pluripotent stem cell” encompasses pluripotent cells, that, like ES cells, can be cultured over a long period of time while maintaining the ability to differentiate into all types of cells in an organism, but that, unlike ES cells (which are derived from the inner cell mass of blastocysts), are derived from differentiated somatic cells, that is, cells that had a narrower, more defined potential and that in the absence of experimental manipulation could not give rise to all types of cells in the organism.

By “having the potential to become an induced pluripotent step cell” it is meant that a differentiated somatic cell can be induced to become, i.e. can be reprogrammed to become, an induced pluripotent step cell. In other words, the somatic cell can be induced to redifferentiate so as to establish cells having the morphological characteristics, growth ability and pluripotency of pluripotent cells. Induced pluripotent step cells have an hESC-like morphology, growing as flat colonies with large nucleo-cytoplasmic ratios, defined borders and prominent nuclei. In addition, induced pluripotent step cells express one or more key pluripotency markers known by one of ordinary skill in the art, including but not limited to AKP, SSEA3, SSEA4, Sox2, Oct3/4, Nanog, TRA-1-60, TRA-1-81, TDGF 1, Dnmt3b, FoxD3, GDF3, Cyp26a1, TERT, and zfp42. In addition, the iPS cells are capable of forming teratomas.

As used herein, the term “pluripotent stem cell induction medium” refers to a medium upon which somatic cells into which suitable proteins (e.g., Oct-3/4, SOX2, c-Myc, and Klf4) or nucleic acids encoding the same can be maintained and/or grown to produce pluripotent stem cells. Recipes for such media are well known in the art and examples are described in Nishimura, et al., J. Biol. Chem, 286: 4760-4771 (2011), US 2010/0279404, US 2011/0250692, US 2011/0287538 and US 2011/0306516.

As used herein, the term “differentiation medium” refers to a medium upon which iPSCs can be grown to become differentiated cells. Recipes for such media are also well known in the art and examples are provided below.

As used herein, the term “differentiate” refers to the development of a cell type that is generic to a more specialized cell type. The term “differentiated” as used herein encompasses cell types that are both partially and terminally differentiated. As used herein the term “differentiate” generally refers to a process by which a generic cell develops into a more specialized cell. A differentiated cell may possess the ability to differentiate into multiple cell lineages and terminally differentiated states. Differentiated cells are cells (other than pluripotent stem cells) derived from pluripotent stem cells. Differentiated cells may be, for example, cells that do not have the ability to differentiate into the three germ layers (the endoderm, mesoderm, and ectoderm). Such cells will not have the ability to form the three germ layers unless they are reprogrammed. Furthermore, differentiated cells may be, for example, cells that cannot produce cells that are not of the germ layer type to which they belong. Differentiated cells may be somatic cells, and for example, they may be cells other than germ cells.

The term “trans-differentiate” or “reprogramming” refers to a differentiation of one somatic cell type to another somatic cell type. This includes (1) a change of the initial somatic cell such as a fibroblast to an iPSC which is then induced to a second somatic cell or (2) direct induction of the second somatic cell from the initial somatic cell.

As used herein, the term “somatic cells” refers to any differentiated cell of a mammal that is not a gamete, germ cell, gametocyte or undifferentiated stem cell. Somatic cells that can be used to produce induced to become pluripotent stem cells include cells from stomach, liver, skin, blood, muscle, bone marrow, umbilical cord blood, spleen, pancreas, lung, intestine, the prostate, stomach, the urinary tract, as well as epithelial cells that are found in urine and other differentiated tissues. These cells may be fresh or frozen cells, which may be from a neonate, a juvenile or an adult. The tissue may be obtained by biopsy or apheresis from a live donor, or obtained from a dead or dying donor within about 48 hours of death, or freshly frozen tissue, tissue frozen within about 12 hours of death. In some cases, the differentiated somatic cells may be human dermal fibroblasts that have been generated from a skin biopsy of a live donor, e.g., an adult human donor. For isolation of cells from tissue, an appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5 mM-25 mM. Convenient buffers include HEPES, phosphate buffers, lactate buffers, etc.

The terms “primary cells”, “primary cell lines”, and “primary cultures” are used interchangeably herein to refer to cells and cells cultures that have been derived from a subject and allowed to grow in vitro for a limited number of passages, i.e. splitting of the culture. For example primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro.

The term “culturing”, in the context of culturing one cell type (e.g., a pluripotent stem cell) into another cell type (e.g., a differentiated cell cell) may be a multistep process.

As used herein, the terms “administering” and “implanting” are intended to encompass direct (e.g., injection directly into a region) and indirect (e.g., systemic administration) methods by which cells are placed in a recipient.

As used herein, the term “inactivated viral particle” refers to an activated, replication defective particle of a virus. Such a particle is incapable of replication in a host cell does not contain a virus genome. Sendai viral particles, for example, are composed of the envelope of the hemagglutinating virus of Japan, i.e., the HVJ envelope. Such particles can be assembled from protein in vitro, e.g., in the presence of detergent. An inactivated Sendai viral particle does not need to contain wild-type HVJ envelope. For example, the particle used may be derived from a natural-occurring strain, a mutant strains, a laboratory-passaged strain, or an artificially constructed strain (see, e.g., US 2013/0210150), and may contain mutant HVJ envelope proteins.

As used herein, the term “packaged within”, in the context of a protein that is packaged within an HVJ envelope or particle, refers to a protein that is packaged in the internal space of an HVJ envelope or particle. As used herein, the term “transfecting” refers to the act of transferring material from inside a viral particle to the inside of a cell. This is done by contacting the viral particle with the surface of the cell.

As used herein, the term “GATA3” refers to GATA-binding protein 3 as well as functionally equivalent man-made variants thereof, e.g., fusion proteins and the like. Genbank accession number CAA38916.1 defines a human GATA3 protein. Orthologs of this protein exist in many other species, and this protein has been characterized both structurally and functionally.

As used herein, the term “NeuroD1” refers to Neurogenic differentiation 1 as well as functionally equivalent man-made variants thereof, e.g., fusion proteins and the like. Genbank accession number AB018693 defines a human NeuroD1protein. Orthologs of this protein exist in many other species, and this protein has been characterized both structurally and functionally.

As used herein, the term “C/EBPβ” refers to CCAAT/enhancer binding protein (C/EBP), beta, as well as functionally equivalent man-made variants thereof, e.g., fusion proteins and the like. Genbank accession number AAN86350.1 defines a human C/EBPβ protein. Orthologs of this protein exist in many other species, and this protein has been characterized both structurally and functionally.

As used herein, the term “Sox2” refers to naturally occurring mammalian SRY (sex determining region Y)-box 2 transcription factors as well as functionally equivalent man-made variants thereof, e.g., fusion proteins and the like. Genbank accession number NP_003097 defines a human Sox2 protein and Genbank accession number NP_035573 defines a mouse Sox2 protein. Orthologs of this protein exist in many other species, and this protein has been characterized both structurally and functionally.

As used herein, the term “Oct4” refers to naturally occurring mammalian octamer-binding transcription factor 4 transcription factor, as well as functionally equivalent man-made variants thereof, e.g., fusion proteins and the like. Genbank accession number NP_001167002 defines a human Oct4 protein and Genbank accession number NP_001239381 defines a mouse Oct4 protein. Orthologs of this protein exist in many other species, and this protein has been characterized both structurally and functionally.

As used herein, the term “Klf4” refers to refers to naturally occurring mammalian Kruppel-like factor 4 transcription factor, as well as functionally equivalent man-made variants thereof, e.g., fusion proteins and the like. Genbank accession number NP_004226 defines a human Klf4 protein and Genbank accession number NP_034767 defines a mouse Klf4 protein. Orthologs of this protein exist in many other species, and this protein has been characterized both structurally and functionally.

As used herein, the term “c-Myc” refers to naturally occurring mammalian bHLH/LZ (basic Helix-Loop-Helix Leucine Zipper) domain transcription factors defined by, e.g., Genbank accession numbers NP_002458 and NP_001170823, as well as functionally equivalent man-made variants thereof, e.g., fusion proteins and the like. Orthologs of this protein exist in many other species, and this protein has been characterized both structurally and functionally.

Other definitions of terms may appear throughout the specification.

### DETAILED DESCRIPTION

As mentioned above, provided herein is an inactivated Sendai viral particle comprising an HVJ envelope and one or more isolated transcription factor proteins selected from, e.g., the group consisting of Sox2, Oct4, Klf4, c-Myc, Oct4, C/EBPβ, GATA3 and NeuroD1 packaged within the particle. As noted above, other transcription factors that are involved in cell differentiation/reprogramming are known and in certain cases may be packaged within a particle instead or in addition to one or more of the transcription facts that are explicitly listed herein.

In some cases, the particle may comprise one of the transcription factor proteins (e.g., Oct4, Sox2, Klf4 or c-Myc), two of the transcription factor proteins (e.g., Sox2 and Oct4, Sox2 and Klf4, Sox2 and c-Myc, Oct4 and Klf4, Oct4 and c-Myc, Klf4 and c-Myc, or Oct4 and C/EBPβ), or three of the transcription factor proteins (e.g., Sox2, Oct4 and Klf4, Sox2, Klf4 and c-Myc, Sox2, Oct4 and c-Myc, Sox2, Oct4 or Klf4 or Sox2, GATA3 and NeuroD1). In certain cases, the particle can contain four of the transcription factors, i.e., Sox2, Oct4, Klf4 and c-Myc. In certain cases, if the particle contains less than four transcription factors then the particle may optionally contain an additional agent, e.g., another protein or a small molecule, which functionally replaces any of the four transcription factors that are not in the particle.

As noted above, any of the packaged transcription factors may have an amino acid sequence that is naturally-occurring. In these embodiments, the transcription factor may or may not be part of a fusion protein that contains, e.g., the transcription factor and an affinity tag (e.g., a V5 tag, a FLAG tag, an HA tag, a myc tag, etc.) that can be used to purify and/or track the transcription factor. In other embodiments, any of the packaged transcription factors may have an amino acid sequence that is non-naturally occurring, i.e., non-wild-type. In these embodiments, at least the DNA binding region of the transcription factors (e.g., the full length protein) may independently have an amino acid sequence that at is at least 70% identical (e.g., at least 80% identical, at least 85% identical, at least 90% identical or at least 95% identical) to the corresponding naturally occurring transcription factor. In these embodiments, the transcription factor may or may not be part of a fusion protein that contains the transcription factor and, e.g., an affinity tag, as described above or the like. The transcription factors used herein do not need to contain a translocation domain, e.g., a Tat translocation sequence or the like. In certain cases, the particle comprises: a) a Sox2 transcription factor has an amino acid sequence that is at least 80% identical to a mammalian, e.g., human or mouse, Sox2 protein and b) an Oct4 transcription factor that has an amino acid sequence that is at least 80% identical to mammalian, e.g., human or mouse, Oct4 protein; c) a Klf4 transcription factor that has an amino acid sequence that is at least 80% identical to mammalian, e.g., human or mouse, Klf4 protein; and d) a c-Myc transcription factor that has an amino acid sequence that is at least 80% identical to mammalian c-Myc protein. Other transcription factors may also have a sequence that is at least 80% identical to a corresponding mammalian transcription factor.

In certain cases, the particle does not detectably contain any unmodified nucleic acid, e.g., unmodified nucleic acid derived from the viral genome, or unmodified nucleic acid encoding any of the transcription factors.

The particles described above may be made by any suitable method. In general terms, the particles may be made by first producing the transcription factors recombinantly, e.g., in a bacterial or yeast host cell, lysing the cells, and then purifying the transcription factors from the cell contents. Methods for expressing and purifying the transcription factors used herein are well known. In certain cases, a transcription factor may be fused with a purification tag (e.g., a HIS tag, MBP, GST or CBP domain, etc.) to facilitate purification of the transcription factor. The purification tag may be cleaved from the transcription factor before the transcription factor is packaged. The transcription factors may be purified until they are at least about 90% pure, e.g., 95% pure, at least 98% or at least or 99% pure, prior to packaging.

In certain cases, a purified transcription factor may be denatured and refolded prior to uses. In these cases, the transcription factor may be denatured by treating the transcription factor with a denaturant (e.g., 6 M guanidine-HCl or 8 M urea), and then slowly decreasing the concentration of the denaturant so that the protein can refold. The concentration of the denaturant may be decreased by slow dilution or dialysis, for example. Methods for denaturing and refolding proteins are well known (see, e.g., De Bernardez Clark, Current Opinion Biotechnol., 9: 157-163 (1998) and Lilie, et al., Current Opinion Biotechnol., 9, 497-501 (1998)).

The inactivated viral particle may be made by packaging the protein inside a viral envelope methods for which are known (see references listed below). An inactivated Sendai viral particle may be made by combining the selected one or more transcription factor proteins with HVJ envelope. HVJ envelope may be obtained by any suitable method, e.g., from chick eggs, inactivated (e.g., using any mutagenic stimulus such as by treatment with a compound, e.g., beta-propiolactone, or by UV irradiation) and purified. In certain cases the HVJ envelope may be purified before it is inactivated. The different transcription factors may or may not be present at the same or similar concentrations. The relative molar ratios of the different transcription factors may be optimized in certain cases. The transcription factors may be independently added to the envelope protein at a concentration in the range of 1 ng/μl to 1 μg/μl, for example.

In certain embodiments, the transcription factor proteins are contacted with HVJ envelope in the presence of a packaging aid, e.g., a detergent, and then optionally centrifuged to purify the particles (which now contain the transcription factors) from unincorporated protein and packaging aid. These packaging methods may be adapted from those described in Kaneda, et al., Mol. Therapy, 6: 219-226 (2002), Kim, et al., Gene Ther., 13:216-24 (2006), Tashiro, Mol. Cell Cardiol., 39:503-9 (2005), Shintankshida, et al., J. Mol. Cell. Cardiol., 53: 233-239 (2012), Shintankshida, et al., Biochim. Biophys. Acta., 1812: 743-751 (2011), Balasubramanian, et al., PLoS One., 5:e11470 (2011) and Kondo, et al., J. Immunol. Methods, 332: 10-17 (2008), as well as others. An HVJ envelope packaging kit is commercially available from Cosmo Bio USA, Inc. (Carlsbad, Calif.). After they are made, the particles may be suspended in a suitable buffer, e.g., PBS, and kept on ice until use.

In some embodiments, the particles described above may be used to produce pluripotent stem cells from somatic cells. These embodiments may involve transfecting somatic cells with an particle as described above, thereby introducing the one or more transcription factor proteins (and any other components that are present in the particles) into the somatic cells. Transfer of the contents of the particles induces the somatic cells to develop into pluripotent stem cells.

In other embodiments, the particles may be used to trans-differentiate a cell type (i.e., convert one somatic cell type into another). In these embodiments, the inactivated viral particle may comprise Oct4 and C/EBPβ and transfection of somatic cells, e.g., fibroblasts, with the viral particle causes the cells to develop into adipocytes. In another example, the inactivated viral particle may comprise Sox2, GATA3 and NeuroD1 and transfection of somatic cells, e.g., fibroblasts, with the viral particle causes the cells to develop into neurons. In certain cases, the transfection may be done by administering the particles to a mammal, e.g., a human, a non-human primate, a mouse, a rat, a cow, a sheep, a goat, a pig, a dog, a cat, or a horse, in vivo. In some embodiments, the particles may be administered to a subject systemically, e.g. through administration into the bloodstream, or locally, through injection directly into or near to a target organ. Local administrations include renal subcapsular, subcutaneous, central nervous system (including intrathecal), intravascular, intrahepatic, intrasplenic, intrasplanchnic, intraperitoneal (including intraomental), or intramuscular administrations. In some embodiments, the administrations are directly into the hepatic duct, or into lymph nodes, bone marrow, and other organs of the body. In some embodiments, administration may be intravenous, intraportal, intrasplanchnic, into the portal vein or hepatic artery, for example.

In other embodiments, the transfecting is done in vitro, i.e., to somatic cells that have been cultured in vitro. The cells may be transfected by the particles several times during cell culture. In these embodiments, the method may comprise culturing the somatic cells on a pluripotent stem cell induction medium, recipes for such media are known in the art (see, e.g., Nishimura, et al., J. Biol. Chem, 286: 4760-4771 (2011), US 2010/0279404, US 2011/0250692, US 2011/0287538 and US 2011/0306516), to produce pluripotent stem cells. The cultured somatic cells used in this embodiment of the method may be obtained from stomach, liver, skin, blood, the prostate, the urinary tract, or urine, for example, although many other somatic cell types can be used. In certain cases, the somatic cells may be isolated from an individual and cultured to produce a culture of primary cells prior to transfection.

At the time of transfection, the concentration of the transcription factors may be in the region of 0.1 ng/ml to 100 ng/ml, e.g., 0.5 ng/ml to 50 ng/ml, although concentrations well outside of these ranges are envisioned. Because of the high efficiency of the method, the number of cells required for transfection may also be low. In certain cases, the method may use as few as 1,000 cells, although up to 10⁴, 10⁵ or 10⁶ or more cells may be transfected under certain circumstances.

The method described herein results in efficient reprogramming of somatic cells into induced pluripotent stem cells, where term “efficiency”, is used to refer to the number of iPSCs produced from a primary cell culture. In certain cases, the somatic cells are reprogrammed to become iPSCs at a rate of at least 10%, at least 20%, at least 30%, at least 50%, at least 70%, at least 80%, or more.

At this point, the pluripotent stem cells may be administered to a mammal, as described above, or transferred to a differentiation medium to cause the induced pluripotent stem cell to differentiate into a differentiated cell type. The cell types that can be produced from pluripotent stem cells, and the culture media that can be used to produce differentiated cells from pluripotent stem cells are numerous and include nerve cells, liver cells, muscle cells, epithelial cells, islet cells, adipose cells, osteoblasts cells, skin cells, red blood cells, white blood cells, to name but a few. Media that causes differentiation of pluripotent stem cells into such differentiated cells are known in the art. Differentiated cells may also be administered to subject, as described above.

Examples of differentiated cells include any differentiated cells from ectodermal (e.g., neurons and fibroblasts), mesodermal (e.g., cardiomyocytes), or endodermal (e.g., pancreatic cells) lineages. The differentiated cells may be one or more: pancreatic beta cells, neural stem cells, neurons (e.g., dopaminergic neurons), oligodendrocytes, oligodendrocyte progenitor cells, hepatocytes, hepatic stem cells, astrocytes, myocytes, hematopoietic cells, or cardiomyocytes. The differentiated cells derived from the induced cells may be terminally differentiated cells, or they may be capable of giving rise to cells of a specific lineage. There are numerous methods of differentiating the induced cells into a more specialized cell type. Methods of differentiating induced cells may be similar to those used to differentiate stem cells, particularly ES cells, MSCs, MAPCs, MIAMI, hematopoietic stem cells (HSCs). In some cases, the differentiation occurs ex vivo; in some cases the differentiation occurs in vivo.

For example, neural stem cells may be generated from ES cells using the method described in, e.g., Reubinoff, et al., Nat, Biotechnol., 19(12): 1134-40 (2001), and the neural stems derived from the induced cells may be differentiated into neurons, oligodendrocytes, or astrocytes. Often, the conditions used to generate neural stem cells can also be used to generate neurons, oligodendrocytes, or astrocytes. Dopaminergic neurons play a central role in Parkinson's disease and other neurodegenerative diseases and are of particular interest. In order to promote differentiation into dopaminergic neurons, induced cells may be co-cultured with a PA6 mouse stromal cell line under serum-free conditions, see, e.g., Kawasaki, et al., Neuron, 28(1):3140 (2000). Other methods have also been described, see, e.g., Pomp, et al., Stem Cells 23(7):923-30 (2005); U.S. Pat. No. 6,395,546, e.g., Lee, et al., Nature Biotechnol., 18:675-679 (2000). Oligodendrocytes may also be generated from the induced cells. Differentiation of the induced cells into oligodendrocytes may be accomplished by known methods for differentiating ES cells or neural stem cells into oligodendrocytes. For example, oligodendrocytes may be generated by co-culturing induced cells or neural stem cells with stromal cells, e.g., Hermann, et al., J Cell Sci., 117(Pt 19):4411-22 (2004). Astrocytes may also be produced from the induced cells. Astrocytes may be generated by culturing induced cells or neural stem cells in the presence of neurogenic medium with bFGF and EGF, see e.g., Brustle et al., Science, 285:754-756 (1999).

Induced cells may be differentiated into pancreatic beta cells by methods known in the art, e.g., Lumelsky, et al., Science, 292:1389-1394 (2001); Assady, et al., Diabetes, 50:1691-1697 (2001); D'Amour, et al., Nat. Biotechnol., 24:1392-1401 (2006); D'Amour, et al., Nat. Biotechnol. 23:1534-1541 (2005). The method may comprise culturing the induced cells in serum-free medium supplemented with Activin A, followed by culturing in the presence of serum-free medium supplemented with all-trans retinoic acid, followed by culturing in the presence of serum-free medium supplemented with bFGF and nicotinamide, e.g., Jiang, et al., Cell Res., 4:333-444 (2007).

Hepatic cells or hepatic stem cells may be differentiated from the induced cells. For example, culturing the induced cells in the presence of sodium butyrate may generate hepatocytes, see e.g., Rambhatla, et al., Cell Transplant, 12:1-11 (2003). In another example, hepatocytes may be produced by culturing the induced cells in serum-free medium in the presence of Activin A, followed by culturing the cells in fibroblast growth factor-4 and bone morphogenetic protein-2, e.g., Cai, et al., Hepatology, 45(5): 1229-39 (2007).

The induced cells may also be differentiated into cardiac muscle cells. Inhibition of bone morphogenetic protein (BMP) signaling may result in the generation of cardiac muscle cells (or cardiomyocytes), see, e.g., Yuasa, et al., Nat. Biotechnol., 23(5):607-11 (2005). In other examples, cardiomyocytes may be generated by culturing the induced cells in the presence of leukemia inhibitory factor (LIF), or by subjecting them to other methods known in the art to generate cardiomyocytes from ES cells, e.g., Bader, et al., Circ. Res., 86:787-794 (2000), Kehat, et al., J. Clin. Invest., 108:407-414 (2001); Mummery, et al., Circulation, 107:2733-2740 (2003).

Examples of methods to generate other cell-types from induced cells include: (1) culturing induced cells in the presence of retinoic acid, leukemia inhibitory factor (LIF), thyroid hormone (T3), and insulin in order to generate adipocytes, e.g., Dani, et al., J. Cell Sci., 110:1279-1285 (1997); (2) culturing induced cells in the presence of BMP-2 or BMP4 to generate chondrocytes, e.g., Kramer, et al., Mech. Dev., 92:193-205 (2000); (3) culturing the induced cells under conditions to generate smooth muscle, e.g., Yamashita, et al., Nature, 408:92-96 (2000); (4) culturing the induced cells in the presence of beta-1 integrin to generate keratinocytes, e.g., Bagutti, et al., Dev. Biol., 179:184-196 (1996); (5) culturing the induced cells in the presence of Interleukin-3 (IL-3) and macrophage colony stimulating factor to generate macrophages, e.g., Lieschke and Dunn, Exp. Hemat., 23:328-334 (1995); (6) culturing the induced cells in the presence of IL-3 and stem cell factor to generate mast cells, e.g., Tsai, et al., Proc. Natl. Acad. Sci. USA, 97:9186-9190 (2000); (7) culturing the induced cells in the presence of dexamethasone and stromal cell layer, steel factor to generate melanocytes, e.g., Yamane, et al., Dev. Dyn., 216:450-458 (1999); (8) co-culturing the induced cells with fetal mouse osteoblasts in the presence of dexamethasone, retinoic acid, ascorbic acid, beta-glycerophosphate to generate osteoblasts, e.g., Buttery, et al., Tissue Eng., 7:89-99 (2001); (9) culturing the induced cells in the presence of osteogenic factors to generate osteoblasts, e.g., Sottile, et al., Cloning Stem Cells, 5:149-155 (2003); (10) overexpressing insulin-like growth factor-2 in the induced cells and culturing the cells in the presence of dimethyl sulfoxide to generate skeletal muscle cells, e.g., Prelle, et al., Biochem. Biophys. Res. Commun., 277:631-638 (2000); (11) subjecting the induced cells to conditions for generating white blood cells; or (12) culturing the induced cells in the presence of BM P4 and one or more: SCF, FLT3, IL-3, IL-6, and GCSF to generate hematopoietic progenitor cells, e.g., Chadwick, et al., Blood, 102:906-915 (2003).

Also provided herein is a screening method in which test agents e.g., proteins (including peptides, gene products, and other transcription factors such as Nanog or Lin-28 and variants thereof), nucleic acids (including DNA or RNA oligonucleotides, regulatory RNAs, inhibitory RNAs, cDNAs, or vectors), or small molecules (e.g., molecules that are up to 500 Da or up to 2500 Da in molecular weight), are tested to determine whether they have any effect on cells produced using the particles. These methods may be adapted from, e.g., Shi, et al., Cell Stem Cell, 3: 568-574 (2008) and US 2011/0014164, among many others.

The resultant cells may be tested for the expression of a variety of markers, including, but not limited to AKP, SSEA3, SSEA4, Sox2, Oct3/4, Nanog, TRA-1-60, TRA-1-81, TDGF 1, Dnmt3b, FoxD3, GDF3, Cyp26a1, TERT, and zfp42. In addition, the iPSCs are capable of forming teratomas. In addition, they are capable of forming or contributing to ectoderm, mesoderm, or endoderm tissues in a living organism.

In these embodiments, the method may comprise: a) transfecting somatic cells with an inactivated viral particle as described above b) contacting a test agent with the somatic cells (which, as mentioned above, may be done before, during or at the same time as the transfection), and c) culturing the somatic cells on a medium such as pluripotent stem cell induction medium (to produce pluripotent stem cells) and, optionally, culturing the pluripotent stem cells on a differentiation medium (to produce pluripotent stem cells). This method further comprises determining whether the test agent has any effect on the cell type produced by culturing step, e.g., any effect on the induction of pluripotent stem cells, or any effect on the differentiation of those cells to a differentiated state. In certain cases, the culturing step c) may comprises culturing the somatic cells on pluripotent stem cell induction medium to produce pluripotent stem cells and, optionally, culturing the pluripotent stem cells on a differentiation medium; and the determining step d) comprises determining whether the test agent has any effect on the differentiation of a second type of somatic cells grown on the differentiation medium, wherein the second type of somatic cells is different to the somatic cells of step b). In certain cases, the test agent, e.g., a protein, may be packaged along with the one or more transcription factor proteins within the inactivated viral particle. In other cases, the test agent is not packaged in the particle and may be added to the medium instead. In these embodiments, the test agent may or may not enter the cell.

Also provided herein is a screening method comprising: a) packaging a test agent, e.g., a nucleic acid, protein or small molecule as described above, within an inactivated viral particle in the absence of isolated Sox2, Oct4, Klf4 and c-Myc proteins or nucleic acid encoding the same; b) transfecting iPSCs with the inactivated viral particle of step a); c) culturing the transfected cells on a differentiation medium and d) determining whether the test agent has any effect on the cell type produced by the culturing step. The various steps of this method can be adapted from the various protocols described above.

The induced cells, or cells differentiated from the induced cells, may be used as a therapy to treat disease (e.g., a genetic defect). The therapy may be directed at treating the cause of the disease; or alternatively, the therapy may be to treat the effects of the disease or condition. The induced cells may be transferred to, or close to, an injured site in a subject, particularly a subject from which the somatic cells were obtained; or the cells can be introduced to the subject in a manner allowing the cells to migrate, or home, to the injured site. The transferred cells may advantageously replace the damaged or injured cells and allow improvement in the overall condition of the subject. In some instances, the transferred cells may stimulate tissue regeneration or repair. The transferred cells may be cells differentiated from induced cells. The transferred cells also may be multipotent stem cells differentiated from the induced cells. In some cases, the transferred cells may be induced cells that have not been differentiated.

The number of administrations of treatment to a subject may vary. Introducing the induced and/or differentiated cells into the subject may be a one-time event; but in certain situations, such treatment may elicit improvement for a limited period of time and require an on-going series of repeated treatments. In other situations, multiple administrations of the cells may be required before an effect is observed. The exact protocols depend upon the disease or condition, the stage of the disease and parameters of the individual subject being treated.

The cells may be introduced to the subject via any of the following routes: parenteral, intravenous, intraarterial, intramuscular, subcutaneous, transdermal, intratracheal, intraperitoneal, or into spinal fluid. The iPSCs produced by the present method can be used to recellularize a decellularized organ or tissue (i.e., a “scaffold”), thereby making an artificial organ or tissue. In these embodiments, the cells can be introduce (i.e., “seeded”) into a decelluralized organ or tissue (e.g., one that has been produced by immersion of an organ or tissue into a detergent composition to detach cellular material from an extracellular matrix) by infusion or injection into one or more locations. Alternatively, or in addition to injection, the induced pluripotent stems can be introduced by perfusion into a cannulated decellularized organ or tissue. In some embodiment, the induced pluripotent stems are perfused into a decellularized organ using a perfusion medium, which can then be changed to an expansion and/or differentiation medium to induce growth and/or differentiation of the induced pluripotent stem cells.

The number of iPSCs that are introduced into and onto a decellularized organ in order to generate an organ or tissue is dependent on both the organ (e.g., which organ, the size and weight of the organ) and other factors. Similarly, different organ or tissues may be cellularized at different densities. By way of example, a decellularized organ or tissue can be seeded with at least about 1,000 (e.g., at least 10,000; 100,000, 1,000,000, 10,000,000, or 100,000,000) induced pluripotent stem cells; or can have from about 1,000 cells/mg tissue (wet weight, i.e., prior to decellularization) to about 10,000,000 cells/mg tissue (wet weight) attached thereto.

During recellularization, an organ or tissue can maintained under conditions in which at least some of the iPSCs can reside, multiply and/or differentiate within and on the decellularized organ or tissue. Those conditions include, without limitation, the appropriate temperature and/or pressure, electrical and/or mechanical activity, force, the appropriate amount of O2 and/or CO2, an appropriate amount of humidity, and sterile or near-sterile conditions. During recellularization, the decellularized organ or tissue and the regenerative cells attached thereto are maintained in a suitable environment. For example, the regenerative cells may require a nutritional supplement (e.g., nutrients and/or a carbon source such as glucose), exogenous hormones or growth factors, and/or a particular pH.

In some embodiments, the exterior surface of the organ or tissue may be bathed in fluid and continually coated with the maintenance solution and/or differentiation medium throughout the recellularization stage to enhance cell viability and differentiation, as well as restructuring of the organ or tissue. The nature and extent of fluid conditions surrounding the organ or tissue can vary according to the specific nature of the organ or tissue. The fluid bathing of the organ or tissue can be intermittent or continuous, partial or complete. As the natural anatomical conduits of the organ or tissue are employed during the recellularization stage, mixtures of excess maintenance solution and regenerative cell medium can exit through natural conduits onto the organ or tissue surface and cover the exterior of the organ or tissue.

The iPSCs can be allogeneic to a decellularized organ or tissue (e.g., a human decellularized organ or tissue seeded with human induced pluripotent stem cells), or regenerative cells can be xenogeneic to a decellularized organ or tissue (e.g., a pig decellularized organ or tissue seeded with human induced pluripotent stem cells). “Allogeneic” as used herein refers to cells obtained from the same species as that from which the organ or tissue originated (e.g., related or unrelated individuals), while “xenogeneic” as used herein refers to cells obtained from a species different than that from which the organ or tissue originated.

In some instances, an organ or tissue generated by the methods described herein is to be transplanted into a patient. In those cases, the iPSCs are used to recellularize a decellularized organ or tissue can be obtained from the patient such that the regenerative cells are “autologous” to the patient.

Irrespective of the source of the cells (e.g., autologous or not), the decellularized organ can be autologous, allogeneic or xenogeneic to a patient. In certain instances, a decellularized organ may be recellularized with cells in vivo (e.g., after the organ or tissue has been transplanted into an individual). In vivo recellularization may be performed as described above (e.g., injection and/or perfusion) with, for example, any of the iPSCs described herein. Alternatively or additionally, in vivo seeding of a decellularized organ or tissue with endogenous cells may occur naturally or be mediated by factors delivered to the recellularized tissue.

Organs and tissues that can be made using this method include, but are not limited to, heart, liver, lung, gall bladder, skeletal muscle, brain, pancreas, spleen, kidney, uterus, and bladder, and portions thereof (e.g., aortic valve, a mitral valve, a pulmonary valve, a tricuspid valve, a pulmonary vein, a pulmonary artery, coronary vasculature, septum, a right atrium, a left atrium, a right ventricle, or a left ventricle, papillary muscle, SA node, or liver lobe, etc.) as well as vasculature (e.g., arteries and veins) of organs and tissues (e.g., heart), bile ducts and veins associated with the liver, ureter of the kidney, trachea of the lung, ventricles of the brain (including lateral ventricles), esophagus of the stomach.

The induced cells may be differentiated into cells and then transferred to subjects suffering from a wide range of diseases or disorders. Subjects suffering from neurological diseases or disorders could especially benefit from stem cell therapies. In some approaches, the induced cells may be differentiated into neural stem cells or neural cells and then transplanted to an injured site to treat a neurological condition, e.g., Alzheimer's disease, Parkinson's disease, multiple sclerosis, cerebral infarction, spinal cord injury, or other central nervous system disorder, see, e.g., Morizane, et al., Cell Tissue Res., 331(1):323-326 (2008); Coutts and Keirstead, Exp. Neurol., 209(2):368-377 (2008); Goswami and Rao, Drugs, 10(10):713-719 (2007).

For the treatment of Parkinson's disease, the induced cells may be differentiated into dopamine-acting neurons and then transplanted into the striate body of a subject with Parkinson's disease. For the treatment of multiple sclerosis, neural stem cells may be differentiated into oligodendrocytes or progenitors of oligodendrocytes, which are then transferred to a subject suffering from MS.

For the treatment of a neurologic disease or disorder, a successful approach may be to introduce neural stem cells to the subject. For example, in order to treat Alzheimer's disease, cerebral infarction or a spinal injury, the induced cells may be differentiated into neural stem cells followed by transplantation into the injured site. The induced cells may also be engineered to respond to cues that can target their migration into lesions for brain and spinal cord repair, e.g., Chen, et al., Stem Cell Rev., 3(4):280-288 (2007).

Diseases other than neurological disorders may also be treated by a stem cell therapy that uses cells differentiated from induced cells, e.g., induced multipotent or pluripotent stem cells. Degenerative heart diseases such as ischemic cardiomyopathy, conduction disease, and congenital defects could benefit from stem cell therapies, see, e.g. Janssens, et al., Lancet, 367:113-121 (2006).

Pancreatic islet cells (or primary cells of the islets of Langerhans) may be transplanted into a subject suffering from diabetes (e.g., diabetes mellitus, type 1), see e.g., Burns, et al., Curr. Stem Cell Res. Ther., 2:255-266 (2006). In some embodiments, pancreatic beta cells derived from induced cells may be transplanted into a subject suffering from diabetes (e.g., diabetes mellitus, type 1).

In other examples, hepatic cells or hepatic stem cells derived from induced cells are transplanted into a subject suffering from a liver disease, e.g., hepatitis, cirrhosis, or liver failure.

Hematopoietic cells or HSCs derived from induced cells may be transplanted into a subject suffering from cancer of the blood, or other blood or immune disorder. Examples of cancers of the blood that are potentially treated by hematopoietic cells or HSCs include: acute lymphoblastic leukemia, acute myeloblastic leukemia, chronic myelogenous leukemia (CML), Hodgkin's disease, multiple myeloma, and non-Hodgkin's lymphoma. Often, a subject suffering from such disease must undergo radiation and/or chemotherapeutic treatment in order to kill rapidly dividing blood cells. Introducing HSCs derived from induced cells to these subjects may help to repopulate depleted reservoirs of cells.

In some cases, hematopoietic cells or HSCs derived from induced cells may also be used to directly fight cancer. For example, transplantation of allogeneic HSCs has shown promise in the treatment of kidney cancer, see, e.g., Childs, et al., N. Engl. J. Med., 343:750-758 (2000). In some embodiments, allogeneic, or even autologous, HSCs derived from induced cells may be introduced into a subject in order to treat kidney or other cancers.

Hematopoietic cells or HSCs derived from induced cells may also be introduced into a subject in order to generate or repair cells or tissue other than blood cells, e.g., muscle, blood vessels, or bone. Such treatments may be useful for a multitude of disorders.

In some cases, the induced cells are transferred into an immunocompromised animal, e.g., SCID mouse, and allowed to differentiate. The transplanted cells may form a mixture of differentiated cell types and tumor cells. The specific differentiated cell types of interest can be selected and purified away from the tumor cells by use of lineage specific markers, e.g., by fluorescent activated cell sorting (FACS) or other sorting method, e.g., magnetic activated cell sorting (MACS). The differentiated cells may then be transplanted into a subject (e.g., an autologous subject, HLA-matched subject) to treat a disease or condition. The disease or condition may be a hematopoietic disorder, an endocrine deficiency, degenerative neurologic disorder, hair loss, or other disease or condition described herein.

The cells may be administered in any physiologically acceptable medium. They may be provided alone or with a suitable substrate or matrix, e.g. to support their growth and/or organization in the tissue to which they are being transplanted. Usually, at least 1×10⁵ cells will be administered, e.g., 1×10⁶ or more. The cells may be introduced by injection, catheter, or the like. The cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or stromal cells associated with progenitor cell proliferation and differentiation. Validation of cell programming may be achieved by QPCR analysis of reverse transcribed total RNA. Reprogrammed cells may be retrieved from in vitro and/or in vivo contexts where a scaffold may be seeded with cells which have been reprogrammed. The cells can be harvested some days or weeks later to verify that reprogramming has occurred. In this way, reprogrammed fibroblasts were shown to become differentiated into adipocytes in one example and into islets in another example.

All references cited herein, including U.S. Provisional Application No. 61/899,075 filed Nov. 1, 2013, U.S. Provisional Application No. 61/987,774 filed May 2, 2014 and U.S. Provisional Application No. 61/993,751 filed May 15, 2014, are incorporated by reference.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the subject invention.

The following abbreviations may be used in the following description: hemagglutinating virus of Japan envelope (HVJ-E), β-TCP, PGA, PPARγ, C/EBPα, GFAP, and MAP2.

The examples described below employ an inactivated Sendai viral particle to deliver transcription factors to cells. The principle of the method described below may be applied to any other viral particles e.g., herpesvirus, parainfluenza virus and lentivirus particles, which can be assembled in vitro and package isolated proteins.

### Example 1

### Expression, Purification and Refolding of Transcription Activators

A. Oct4 , Sox 2 and c-Myc
Oct4 sequence:
(SEQ ID NO: 1)
MGSSHHHHHHSSGLVPRGSHMASMTGGQQMGRGSEMAGHLASDFAFSPPP
GGGGDGPGGPEPGWVDPRTWLSFQGPPGGPGIGPGVGPGSEVWGIPPCPP
PYEFCGGMAYCGPQVGVGLVPQGGLETSQPEGEAGVGVESNSDGASPEPC
TVTPGAVKLEKEKLEQNPEESQDIKALQKELEQFAKLLKQKRITLGYTQA
DVGLTLGVLFGKVFSQTTICRFEALQLSFKNMCKLRPLLQKWVEEADNNE
NLQEICKAETLVQARKRKRTSIENRVRGNLENLFLQCPKPTLQQISHIAQ
QLGLEKDVVRVWFCNRRQKGKRSSSDYAQREDFEAAGSPFSGGPVSFPLA
PGPHFGTPGYGSPHFTALYSSVPFPEGEAFPPVSVTTLGSPMHSNHHHHH
H
Oct4-flag sequence
(SEQ ID NO: 2)
MGSSHHHHHHSSGLVPRGSHMDYKDDDDKAGHLASDFAFSPPPGGGGDGP
GGPEPGWVDPRTWLSFQGPPGGPGIGPGVGPGSEVWGIPPCPPPYEFCGG
MAYCGPQVGVGLVPQGGLETSQPEGEAGVGVESNSDGASPEPCTVTPGAV
KLEKEKLEQNPEESQDIKALQKELEQFAKLLKQKRITLGYTQADVGLTLG
VLFGKVFSQTTICRFEALQLSFKNMCKLRPLLQKWVEEADNNENLQEICK
AETLVQARKRKRTSIENRVRGNLENLFLQCPKPTLQQISHIAQQLGLEKD
VVRVWFCNRRQKGKRSSSDYAQREDFEAAGSPFSGGPVSFPLAPGPHFGT
PGYGSPHFTALYSSVPFPEGEAFPPVSVTTLGSPMHSNHHHHHH
Sox2 sequence:
(SEQ ID NO: 3)
MYNMMETELKPPGPQQTSGGGGGNSTAAAAGGNQKNSPDRVKRPMNAFMV
WSRGQRRKMAQENPKMHNSEISKRLGAEWKLLSETEKRPFIDEAKRLRAL
HMKEHPDYKYRPRRKTKTLMKKDKYTLPGGLLAPGGNSMASGVGVGAGLG
AGVNQRMDSYAHMNGWSNGSYSMMQDQLGYPQHPGLNAHGAAQMQPMHRY
DVSALQYNSMTSSQTYMNGSPTYSMSYSQQGTPGMALGSMGSVVKSEASS
SPPVVTSSSHSRAPCQAGDLRDMISMYLPGAEVPEPAAPSRLHMSQHYQS
GPVPGTAINGTLPLSHMHHHHHH
c-Myc sequence
(SEQ ID NO: 4)
MASMTGGQQM GRGSEFMDFF RVVENQQPPA TMPLNVSFTN
RNYDLDYDSV QPYFYCDEEE NFYQQQQQSE LQPPAPSEDI
WKKFELLPTP PLSPSRRSGL CSPSYVAVTP FSLRGDNDGG
GGSFSTADQL EMVTELLGGD MVNQSFICDP DDETFIKNII
IQDCMWSGFS AAAKLVSEKL ASYQAARKDS GSPNPARGHS
VCSTSSLYLQ DLSAAASECI DPSVVFPYPL NDSSSPKSCA
SQDSSAFSPS SDSLLSSTES SPQGSPEPLV LHEETPPTTS
SDSEEEQEDE EEIDVVSVEK RQAPGKRSES GSPSAGGHSK
PPHSPLVLKR CHVSTHQHNY AAPPSTRKDY PAAKRVKLDS
VRVLRQISNN RKCTSPRSSD TEENVKRRTH NVLERQRRNE
LKRSFFALRD QIPELENNEK APKVVILKKA TAYILSVQAE
EQKLISEEDL LRKRREQLKH KLEQLRNSCA HHHHHH

Oct4 and Sox2 genes were inserted into pET28a (EMD Millipore, Billerica, Mass.) while c-Myc was inserted into pET21a (EMD Millipore, Billerica, Mass.). The engineered plasmids were transformed into separate Rosette™ DE3 (EMD Millipore, Billerica, Mass.) preparations and grown on LB/Kan plates using standard protocols. In each case, a single clone was selected and cultivated on selective media using standard techniques. Cells were harvested from a 1 liter culture. An insoluble pellet of Oct4, Oct4-flag, sox-2 or c-myc proteins was obtained. In each case, a pellet was solubilized and the protein initially purified on a nickel column.

The denatured protein eluate was diluted 10-fold in pre-cooled refolding buffer (50 mM Tris.Cl, pH 8.5, 500 mM NaCl, 500 mM Arg, 0.1% PEG4000, 0.1 mM EDTA, 1 mM GSH and 0.1 mM GSSH). The refolded protein was obtained at a final concentration 0.05-0.1 mg/ml. After a further step of dialysis, the protein was again loaded on a nickel column and eluted with 100% buffer B (Buffer A: 1×PBS buffer, 5 mM β-ME, 5% glycerol and 10 mM imidazole; Buffer B: 1×PBS buffer, 5 mM β-ME, 5% glycerol and 500 mM imidazole) followed again by dialysis against the storage buffer (1×PBS buffer, 5 mM DTT and 50% glycerol). The protein was stored at −80° C. avoiding repeated freeze thaw cycles.

B. Klf4 in Fibroblasts or Bacteria
Klf4 sequence:
(SEQ ID NO: 5)
MRQPPGESDMAVSDALLPSFSTFASGPAGREKTLRQAGAPNNRWREELSH
MKRLPPVLPGRPYDLAAATVATDLESGGAGAACGGSNLAPLPRRETEEFN
DLLDLDFILSNSLTHPPESVAATVSSSASASSSSSPSSSGPASAPSTCSF
TYPIRAGNDPGVAPGGTGGGLLYGRESAPPPTAPFNLADINDVSPSGGFV
AELLRPELDPVYIPPQQPQPPGGGLMGKFVLKASLSAPGSEYGSPSVISV
SKGSPDGSHPVVVAPYNGGPPRTCPKIKQEAVSSCTHLGAGPPLSNGHRP
AAHDFPLGRQLPSRTTPTLGLEEVLSSRDCHPALPLPPGFHPHPGPNYPS
FLPDQMQPQVPPLHYQELMPPGSCMPEEPKPKRGRRSWPRKRTATHTCDY
AGCGKTYTKSSHLKAHLRTHTGEKPYHCDWDGCGWKFARSDELTRHYRKH
TGHRPFQCQKCDRAFSRSDHLALHMKRHFLESRGPFEQKLISEEDLNMHT
EHHHHHH

(i) In Fibroblasts

Fibroblasts (FreeStyle™ 293-F cells (Life Technologies, Carlsbad, Calif.) were transfected with pcDNA 3.1 (Life Technologies, Carlsbad, Calif.) engineered to contain Klf4 using standard methods described by the vendor and obtained Klf4 protein. Harvested cells were lysed and the supernatant was filtered through a 0.22 μM membrane and loaded onto a 5-ml DEAE column. The flow-through was collected and loaded onto a 1 ml Nickel column. The protein was eluted by means of an elution Buffer (50 mM Tris.Cl, pH 7.3, 150 mM NaCl, 250 mM Imidazole) and the eluent dialyzed into storage buffer (20 mM Tris.Cl pH 8, 1 mM DTT, 100 mM NaCl, 50% Glycerol) and stored at −80° C.

(ii) In Bacteria

Klf4 sequence:
(SEQ ID NO: 6)
MRQPPGESDMAVSDALLPSFSTFASGPAGREKTLRQAGAPNNRWREELSH
MKRLPPVLPGRPYDLAAATVATDLESGGAGAACGGSNLAPLPRRETEEFN
DLLDLDFILSNSLTHPPESVAATVSSSASASSSSSPSSSGPASAPSTCSF
TYPIRAGNDPGVAPGGTGGGLLYGRESAPPPTAPFNLADINDVSPSGGFV
AELLRPELDPVYIPPQQPQPPGGGLMGKFVLKASLSAPGSEYGSPSVISV
SKGSPDGSHPVVVAPYNGGPPRTCPKIKQEAVSSCTHLGAGPPLSNGHRP
AAHDFPLGRQLPSRTTPTLGLEEVLSSRDCHPALPLPPGFHPHPGPNYPS
FLPDQMQPQVPPLHYQELMPPGSCMPEEPKPKRGRRSWPRKRTATHTCDY
AGCGKTYTKSSHLKAHLRTHTGEKPYHCDWDGCGWKFARSDELTRHYRKH
TGHRPFQCQKCDRAFSRSDHLALHMKRHFHHHHHH

Klf4 genes were inserted into pET28a while c-Myc was inserted into pET21a. The purification of Klf4 followed the protocol described above for Oct4, Sox2 and c-Myc.

In all cases, quality control to exclude endonuclease, exonuclease and endotoxins were performed using standard assays (See New England Biolabs, Inc. catalog 2013/14). A protease inhibition step was optionally added using for example PMSF (Thermo Scientific, Waltham, Mass.).

### DNA Binding Activity Assay

DNA-binding activity was tested with a standard electrophoresis mobility shift assay (EMSA) using SYBR® (Life Technologies, Carlsbad, Calif.) to detect the bands on polyacrylamide gels.

The following complementary sequences were used:

Specific DNA fragments for Sox2:
(SEQ ID NO: 7)
GAGACTTAATAACAAAGACCTGAAGCAGAGTCAG
Specific DNA fragments for Oct4:
(SEQ ID NO: 8)
CTCGAGACTTAATAATTTGCATACCCTGAAGGCAGGAGTCAG
Specific DNA fragments for c-Myc:
(SEQ ID NO: 9)
CTCGAGACTTAATACACGTGACCTGAAGGCAGAGTCAG
Specific DNA fragments for Klf4:
(SEQ ID NO: 10)
CTGACTCTGCCTTCAGGTCACCCTATTAAGTCTCGAG

### Example 2

### Procedure for Fibroblasts Reprogramming

Transduction of Transcription Factors into Fibroblasts

Human foreskin fibroblast cells were transduced with transcription factors using HVJ-E 4 proteins: Oct4 (8×10-3 μg/ml); Sox2 (8×10-3 μg/ml); c-Myc (8×10-3 μg/ml) and Klf4 (8×10-3 μg/ml) made according to the description above were packaged into freeze-dried HVJ-envelopes using GenomONE™-NeoEX HVJ Envelope Vector KIT (Cosmo Bio USA, Carlsbad, Calif.) using the protocol provided by the manufacturer.

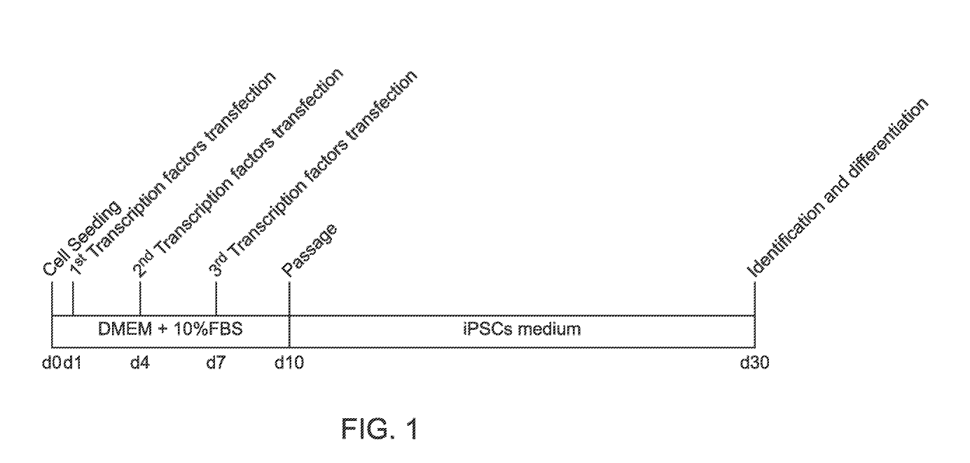

A summary of the timeline for fibroblast reprogramming is shown in FIG. 1. On day 1, transcription factors were transduced into fibroblasts by HVJ-E, and a day later, the medium was replaced and cells cultured. A second transduction using the HVJ-E occurred on Day 4 and a third transduction occurred on day 7. On day 10, the transduced cells were plated on mitomycin C (MMC) treated MEF culture dishes (Applied Stemcell Inc, CA) with 2×105 cells per dish and incubate at 37° C., 5% CO₂. The medium was changed to a serum free medium identified as KnockOut™ (Life Technology, Carlsbad, Calif.) more specifically KnockOut DMEM/F12, 20% KnockOut™ Serum Replacement, 100 μM MEM Non-Essential Amino Acids Solution, 1×GlutaMAX™-I Supplement, 100 μM β-mercaptoethanol, 1×Penicillin-Streptomycin, 4 ng/ml Basic FGF. From Day 11 to 30, the cells were fed and monitored and the culture medium changed every day.

### Flow Cytometry Analysis of HVJ-E Treated Fibroblasts

Human fibroblasts were incubated in 24-well plates at a density of 5×104 cells/well, cultured in DMEM with GlutaMAX™ (high glucose) (Life Technologies, Carlsbad, Calif.) containing 10% fetal bovine serum until the cells fusion reach 90%. The fibroblasts were transfected with HVJ-E without proteins, cells incubated at 37° C. under 5% CO₂ (medium renewed as needed) overnight. 200 μl 0.25% Trypsin/EDTA was added to each well and incubated until the cells have become detached. Washed cell suspension was transferred to a sterile Universal container, centrifuged and the pellet resuspended in 1 ml PBS/10% FCS. The total number of cells was counted using a hemocytometer. 50 μl of appropriately diluted primary antibody was added to a cell suspension of 1×106 cells/ml. The antibodies used here were: Anti-CD105 antibody (Abcam, Cambridge, Mass.) 1:200; Anti-CD24 antibody (Abcam, Cambridge, Mass.) 1:100; Anti-CD90 antibody (Abcam, Cambridge, Mass.) 1:100; and Anti-SSEA3 antibody (Abcam, Cambridge, Mass.) 1:500. After a 60 minute incubation at 4° C. the cells were washed, centrifuged, stained with 100 μl of a second antibody selected from Anti-mouse IgG(H+L) Alexa Fluor® 488 Conjugate (Life Technologies, Carlsbad, Calif.) 1:1000 and Anti-rat IgG(H+L) Alexa Fluor 488 Conjugate 1:1000. The stained cell pellets were resuspended in 200 μl PBS/10% FCS at 4° C. for flow cytometry analysis.

### Location of the Transfected Transcription Factors

To determine the intracellular localization of transcription factors, 24 hours, 48 hours, 72 hours and 96 hours post transduced with proteins, cells were washed with PBS, fixed in 4% (v/v) formaldehyde in PBS at room temperature for 1 minute. Cells were washed in PBS and incubated in the diluted primary antibody His-Tag® Monoclonal Antibody (Novagen, Madison, Wis.) 1:1000 at room temperature for 1 hour. After washing the cells with PBS, fluorescence conjugated secondary antibody (Anti-mouse IgG(H+L)) Alexa Fluor 488 1:1000 was added and incubated for 1 hour at room temperature in the dark. Cells were again washed with PBS and labeled nuclear DNA by Hoechst 33342 stain (Sigma-Aldrich, St. Louis, Mo.) 1:3000. After been washed with PBS cells were immunoreactions observed by fluorescence microscopy.

### Example 3

### Validation of Fibroblasts Reprogramming Protocol

### Flow Cytometry Analysis

Using a similar protocol to that described above, 50 μl of appropriately diluted primary antibody: Anti-CD105 antibody 1:200; Anti-CD24 antibody 1:100; Anti-CD90 antibody 1:1; Anti-SSEA3 antibody 1:500; Anti-Oct4 antibody (Santa Cruz Biotechnology, Dallas, Tex.) 1:100; and Anti-Sox2 antibody (Abcam, Cambridge, Mass.) 1:100, was added to a 50 μl of a single cell suspension in PBS/10% FCS at a concentration of 1×106 cells/ml. The cells were washed, centrifuged and resuspended in 100 μl of diluted second antibody (Anti-mouse IgG(H+L) Alexa Fluor 488 Conjugate 1:1000). The cells were washed, centrifuged and resuspended in 200 μl PBS/10% FCS at 4° C. for flow cytometry analysis.

### Immunofluorescence Staining

Cells were fixed in 4% (v/v) formaldehyde in PBS at room temperature for 30 minutes. For removal of nonspecific binding of the antibodies, cells were incubated with 1% BSA overnight at 4° C. After washing in PBS, cells were incubated in the diluted primary antibody at room temperature for 2 hours. Anti-CD105 antibody 1:200; Anti-CD24 antibody 1:100; Anti-CD90 antibody 1:100; Anti-SSEA3 antibody 1:500; Anti-TRA-1-60 antibody (EMD Millipore, Billerica, Mass.) 1:100; AntiTRA-1-81 antibody (EMD Millipore, Billerica, Mass.) 1:100; and Anti-Sox2 antibody 1:100. Again the cells were washed with PBS. Fluorescence conjugated secondary antibody Anti-mouse IgG(H+L) Alexa Fluor 488 Conjugate 1:1000; and Anti-rat IgG(H+L) Alexa Fluor 488 Conjugate 1:1000 was added and incubated for 1 hour at room temperature in the dark. The cells were again washed with PBS and nuclear DNA was labeled by Hoechst 33342 stain. After been washed with PBS the immunoreactions were viewed with a fluorescence microscope.

### Alkaline Phosphatase (AKP) Activity Assay

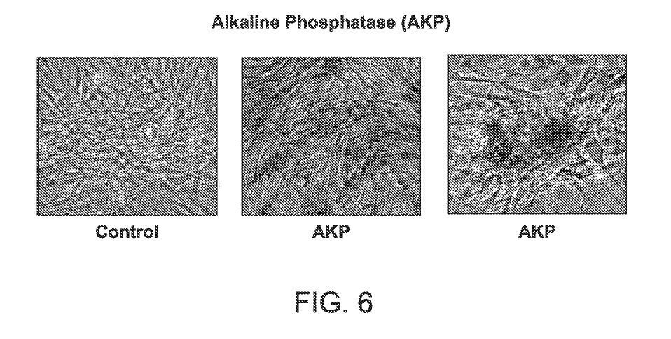

On day 30, cell media was aspirated and the reprogrammed fibroblasts were fixed and prepared for AKP staining (SCR004, (EMD Millipore, Billerica, Mass.)) using the manufacturer's instructions. Fast Red Violet solution: NaphtholAS-BI phosphate solution: Water 2:1:1. The AKP positive cells were observed under the microscope (see FIG. 6).

Q-PCR Analysis iPSCs were sorted by Anti-CD24 by BD FACSAria II™ cell sorting system (BD Biosciences, San Jose, Calif.), cell staining protocol see above.

Total RNA was isolated using TRIZoI® Reagent (Life Technologies, Carlsbad, Calif.) followed by cDNA synthesis using M-MuLV Reverse Transcriptase and Oligo (dT)23VN (New England Biolabs, Ipswich, Mass.). Q-PCR was performed with SsoAdvanced™ Universal SYBR Green (Bio-Rad, Hercules, Calif.).

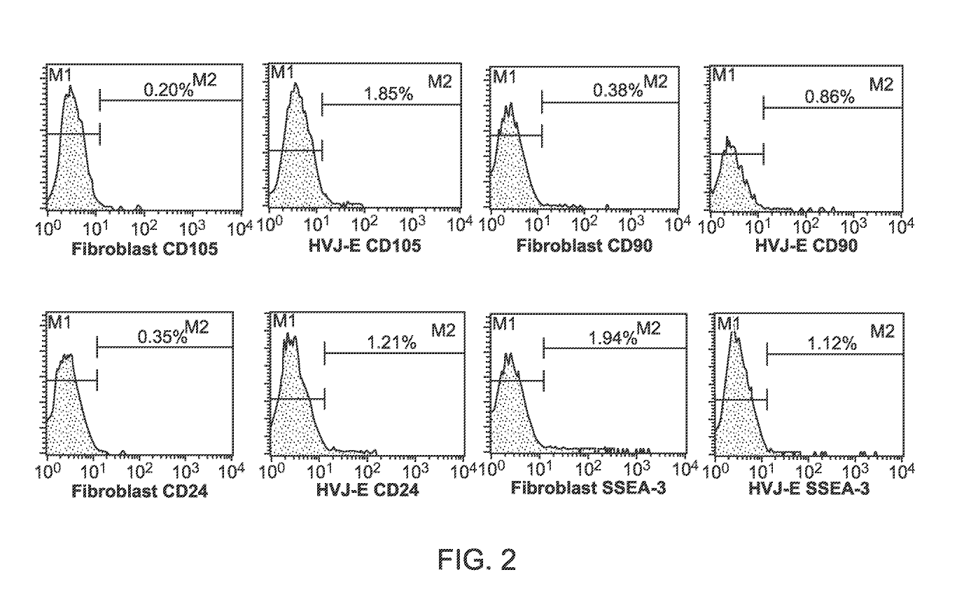

Results from Examples 1-3: The results obtained in this example are shown in FIGS. 2-8. FIG. 2 shows that fibroblasts cell markers expression do not change significantly after the cells have been treated with HVJ-E without proteins. HVJ-E treatment does not affect cell properties of the fibroblast cells.

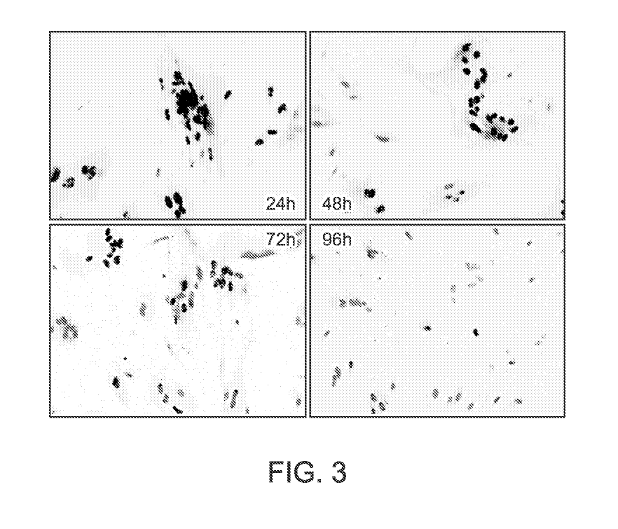

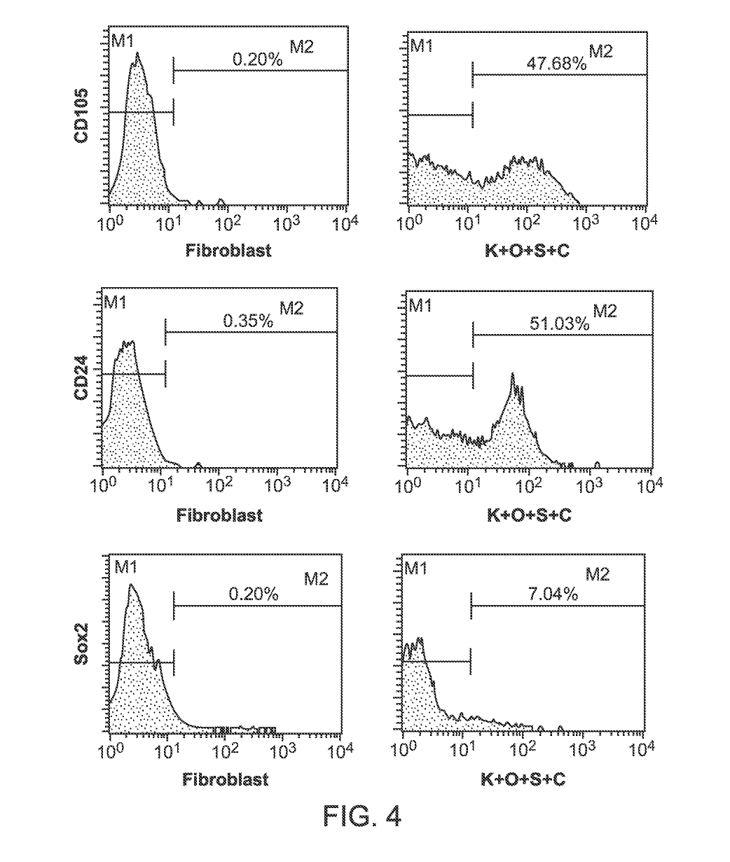

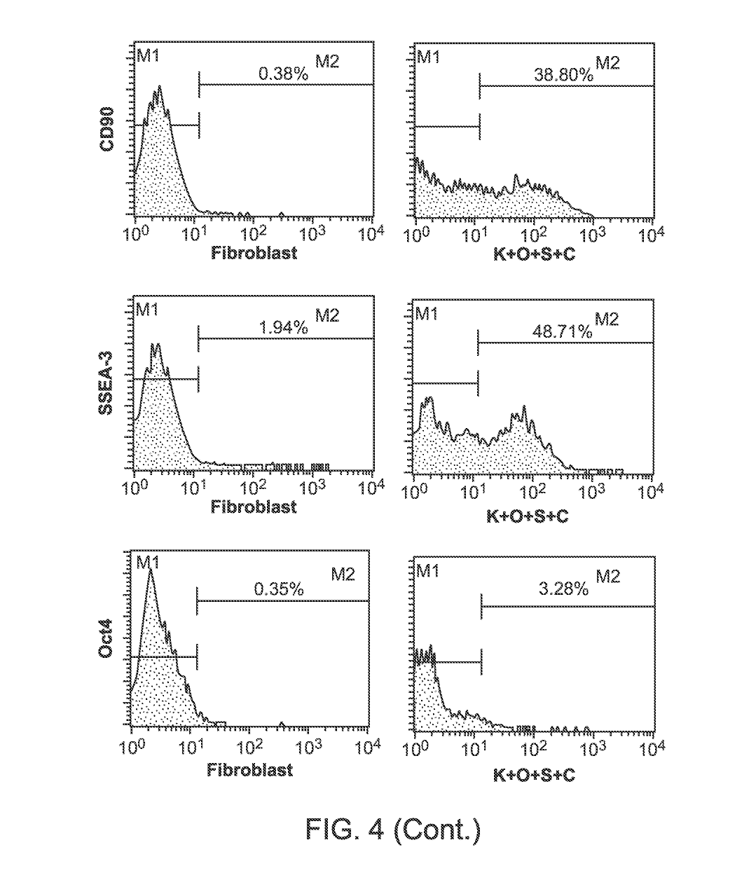

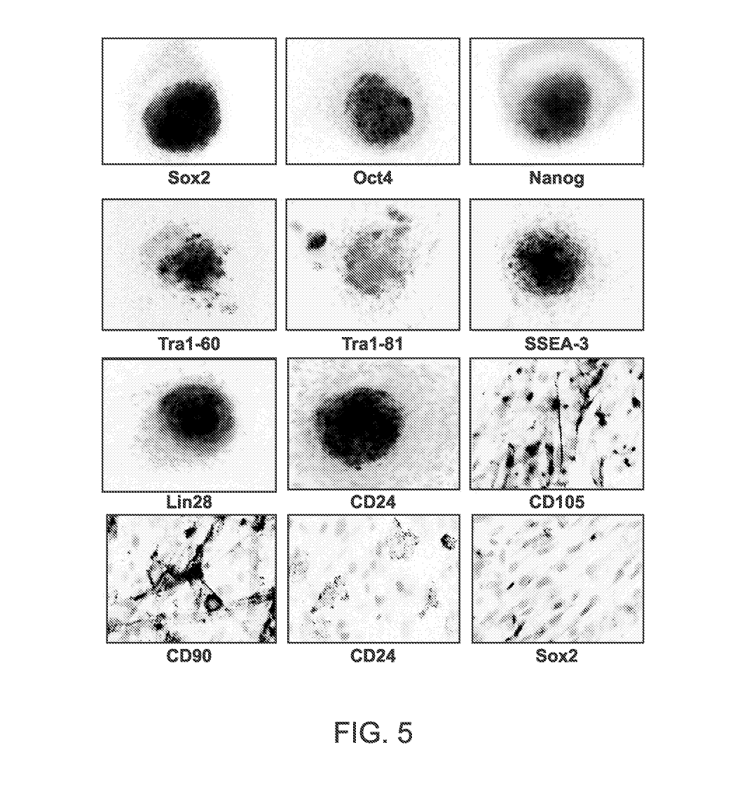

FIG. 3 shows that transfected proteins were localized by anti-His-tag antibody, 24 hour after transfection 90% living cells were positive for His-tag transcription factors, and they all localized in the nucleus. During the time course fluorescence intensity decreased, 96 hours after transfection there is only 5% of the fibroblasts were positive fluorescence. We can conclude that HVJ-E could transfect the proteins into fibroblasts, and transfected transcription factors remain in the nucleus of fibroblasts for at least 72 hours. FIG. 4 shows the results of flow cytometry analysis of the induced iPSCs. After having been transfected with all the four transcription factors (Oct4 (O), Sox2 (S), Klf4 (K) and c-Myc (C)), the stem cell surface marker all increase significantly this confirms that stem cells have been generated. The expression of Sox2 and Oct4 also increased slightly after reprogramming. In FIG. 5 immunofluorescence confirms that the expression of stem cell markers in induced iPSCs. In FIG. 6, it can be seen that alkaline phosphatase-positive colony formation is a sensitive, specific and quantitative indicator for undifferentiated human embryonic stem cells. After reprogrammed by transcription factors, the AKP activity increased significantly, resulting in AKP positive colonies.

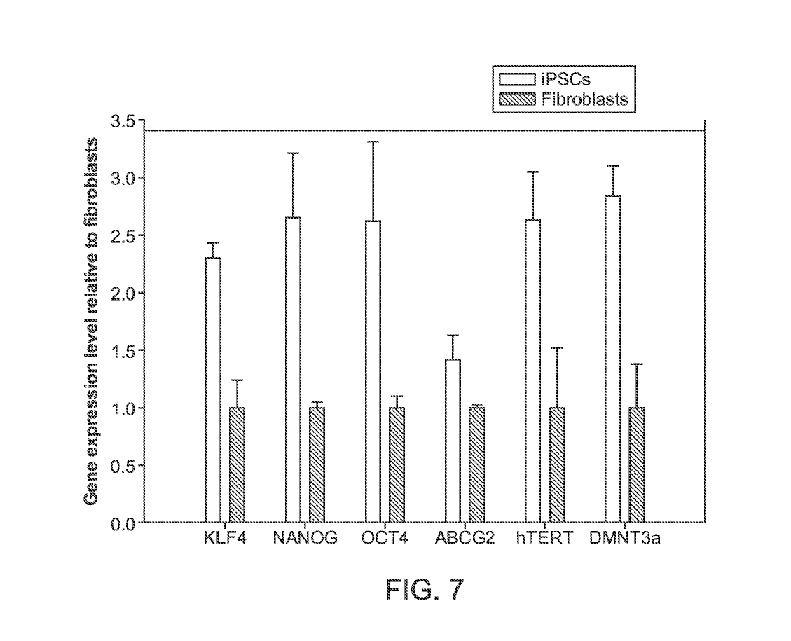

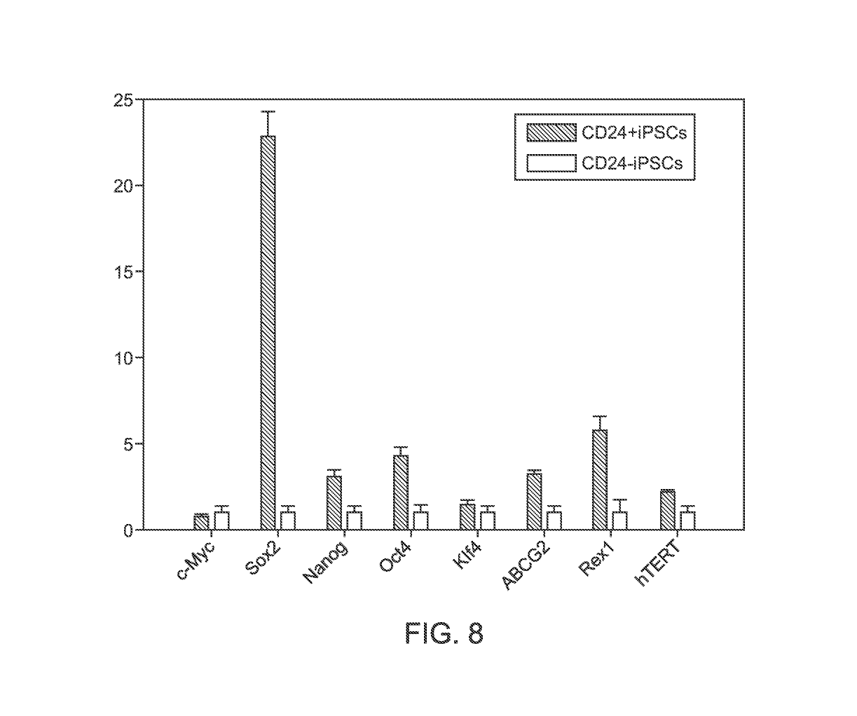

FIG. 7 shows the results of gene expression levels of Klf4, Nanog, Oct4, ABCG2, hTERT and DMNT3a after analysis of mRNA using quantitative RT-PCR in induced iPSCs compared with fibroblasts without reprogramming. The expression level increased significantly compared with control group. FIG. 8 shows gene expression levels of c-Myc, Sox2, Nanog, Oct4, Klf4, ABCG2, Rex1 and hTERT after analysis of mRNA by quantitative RT-PCR in CD24 positive induced iPSCs and in CD24 negative iPSCs. The expression levels of these transcription factors increased significantly in CD24 positive groups.

### Example 4

### Identification of iPSCs Pluripotent Potential In Vitro

Adipogenic Differentiation of iPSCs and Identification of Induced Cells In Vitro

iPSCs were treated with adipogenic induction medium (10% FBS/DMEM, 500 μM IBMX, 1 μM Dexamethasone, 10 μg/ml Insulin, 200 μM Indomethacin) over a 3 week period, with media changed every third day. During that time, iPSCs were induced to form adipogenic cells. The induced cells were analyzed as follows:

Staining: 0.5% Oil Red 0 solution (Sigma-Aldrich, St. Louis, Mo.) was used to stain the cells using the manufacturer's instructions.

RT-PCR: Total RNA was isolated using TRIZol Reagent followed by cDNA synthesis using M-MuLV Reverse Transcriptase and Oligo (dT)23VN. PCR was performed with LongAmp® Taq DNA polymerase (New England Biolabs, Ipswich, Mass.).

Q-PCR analysis: Total RNA was isolated using TRIZoI® Reagent followed by cDNA synthesis using M-MuLV Reverse Transcriptase and Oligo (dT)23VN (NEB). Q-PCR was performed with SsoAdvanced Universal SYBR Green.

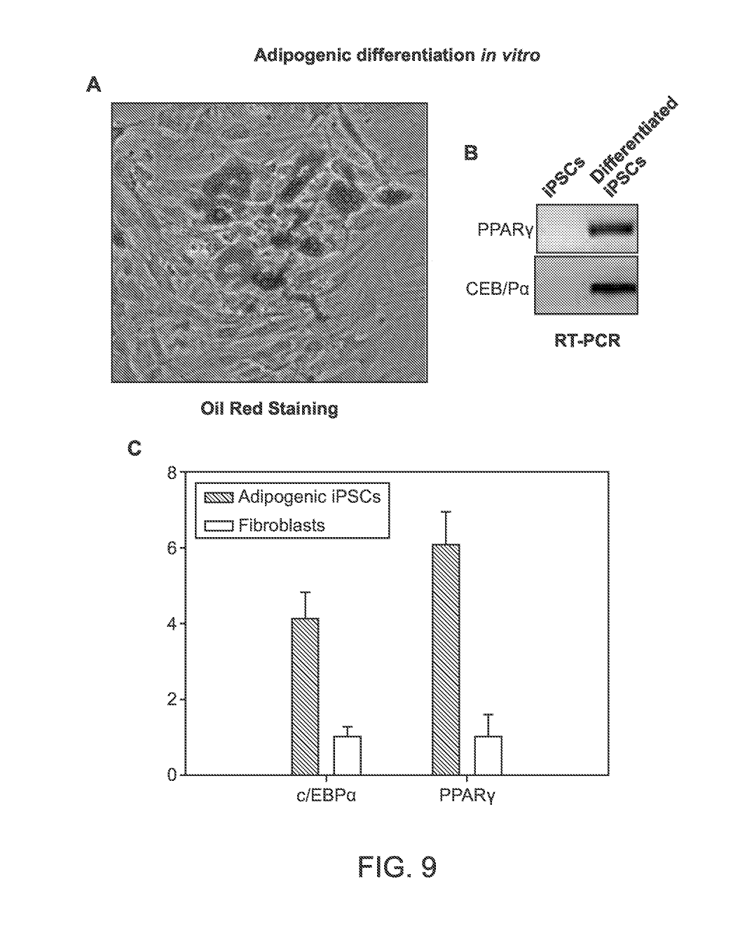

The results showed that iPSCs could be induced into adipocytes in medium containing 1 μM Dexamethasone, 200 μM Indomethacin, 500 μM IBMX and 10 μg/ml Insulin. Red oil drops were stained by Oil Red (FIG. 9A). The expression of PPARy and C/EBPa was induced in the adipogenic group (FIG. 9B). The expression level of PPARy and C/EBPa increased significantly compared with control group (FIG. 9C).

Osteogenic Differentiation from iPSCs In Vitro

iPSCs were treated with osteogenic induction medium (10% FBS/DMEM, 50 μM L-ascorbic acid, 10 mM β-glycerophosphate, 0.1 μM Dexamethasone) with media changed every third day was added to iPSCs (tranduced fibroblasts) for 2 week to induce osteogenic cells. The cells were analyzed by immunofluorescence staining and RT-PCR.

The induced cells were analyzed using immunofluorescence staining of osteocalcin using osteocalcin Antibody (G-5) (Santa Cruz Biotechnology, Dallas, Tex.) 1:200 using the protocol described above. Alizarin Red S staining (Sigma-Aldrich, St. Louis, Mo.) was used to stain calcium compounds using a protocol of the manufacturer. RT-PCR was performed as described above.

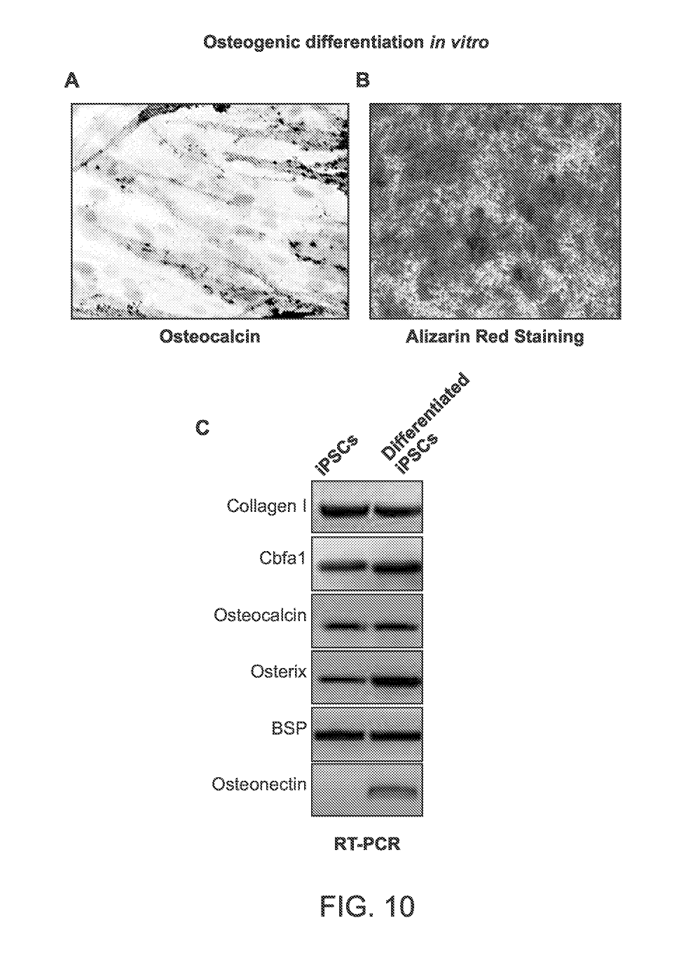

The results showed that iPSCs could be induced into osteoblasts by the media with 50 μM L-ascorbic acid, 10 mM β-glycerophosphate and 0.1 μM dexamethasone. iPSCs were defined as osteoblast-like cells by the staining of osteocalcin (FIG. 10A). Alizarin Red staining showed the calcium nodules secreted by induced osteoblasts (FIG. 10B). Except for osteonectin, specific genes of osteoblasts were expressed both in iPSCs and osteogenic groups (FIG. 10C).

Neurogenic Differentiation and Identification of iPSCs In Vitro

iPSCs were treated with neurogenic induction medium: (10% FBS/DMEM, 5 mM KCl, 2 μM Valproic acid (Sigma-Aldrich, St. Louis, Mo.), 10 μM Forskolin (Sigma-Aldrich, St. Louis, Mo.), 1 μM Hydrocortisone (Sigma-Aldrich, St. Louis, Mo.), 5 μg/ml Insulin (Sigma-Aldrich, St. Louis, Mo.)) which were induced for 1 week with media changed every third day to form neurogenic cells.

The induced cells were analyzed as follows:

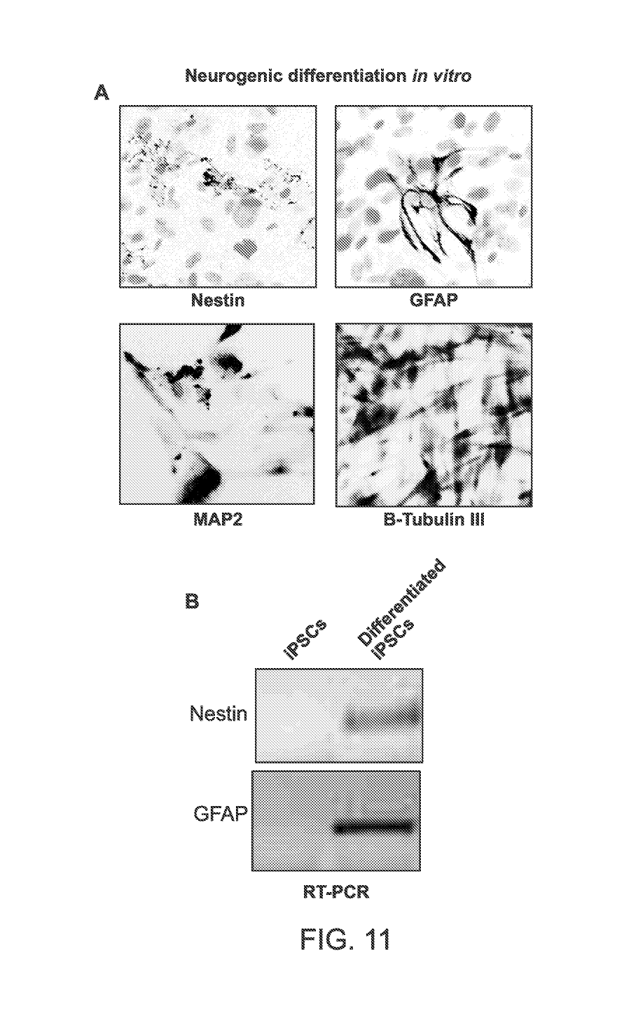

Immunofluorescence staining: Cells were incubated in the diluted primary antibody: Anti-GFAP antibody (Cell Signaling Technology, Danvers, Mass.) 1:300, Anti-Nestin antibody (Cell Signaling Technology, Danvers, Mass.) 1:300, Anti-MAP2 antibody (Abcam, Cambridge, Mass.) 1:200 and Anti-β-Tublin III (Abcam, Cambridge, Mass.) 1:200 at room temperature for 2 hours. Cells were then reacted after washing with Fluorescence conjugated secondary antibody Anti-mouse IgG (H+L) Alexa Fluor 488 Conjugate 1:1000, Anti-rat IgG (H+L) Alexa Fluor 488 Conjugate 1:1000 and Anti-rabbit IgG(H+L) Alexa Fluor 488 Conjugate 1:1000. Nuclear DNA was labeled by Hoechst 33342. The results are shown in FIG. 11A-B.

RT-PCR was performed as described above.

The results showed that the iPSCs could be induced into neurons in media containing 5 mM KCl, 2 μM Valproic acid, 10 μM Forskolin, 1 μM hydrocortisone and 5 μg/ml insulin. The induced cells showed typical neuron morphology with soma, dendrites and axon. GFAP, Nestin, MAP2 and β-Tubulin III were identified by immunofluorescence staining, while GFAP and Nestin expression was confirmed by RT-PCR analysis.

Pancreatic Differentiation and Identification of iPSCs In Vitro

Pancreatic differentiation was induced by culturing in the presence of the following meida and additives: Day 1: RPMI (without FBS) (Life Technologies, Carlsbad, Calif.), activin A (100 ng/ml) (R&D Systems, Minneapolis, Minn.) and Wnt3a (25 ng/ml) (R&D Systems, Minneapolis, Minn.); Day 2 to Day 3: RPMI with 0.2% vol/vol FBS and activin A (100 ng/ml) Day 4 to Day 6: RPMI with 2% vol/vol FBS and FGF-10 (50 ng/ml) (R&D Systems, Minneapolis, Minn.); Day 7 to Day 9: DMEM with 1% vol/vol B27 supplement (Life Technologies, Carlsbad, Calif.), Cyclopamine (0.25 μM) (Sigma-Aldrich, St. Louis, Mo.), all-trans retinoic acid (RA, 2 μM) (Sigma-Aldrich, St. Louis, Mo.) and Noggin (50 ng/ml) (R&D Systems, Minneapolis, Minn.); Day 10 to Day 12: DMEM with 1% vol/vol B27 supplement.

The induced cells were analyzed using immunofluorescence staining and a glucose stimulated insulin secretion assay.

Immunofluorescence staining: Primary antibody at room temperature for 2 hours: Anti-Pdx1 antibody (Cell Signaling Technology, Danvers, Mass.) 1:400; Anti-Glucagon antibody (Cell Signaling Technology, Danvers, Mass.) 1:400; Anti-Insulin antibody (Cell Signaling Technology, Danvers, Mass.) 1:400; Fluorescence conjugated secondary antibody: Anti-rabbit IgG-PE (Santa Cruz Biotechnology, Dallas, Tex.) 1:1000; Anti-rabbit IgG Fab2 Alexa Fluor 488 Conjugate 1:1000; Label nuclear DNA by Hoechst 33342. The immunoreactions were observed by fluorescence microscopy.

Glucose stimulated insulin secretion assay: To determine whether cells could respond to glucose in vitro, the differentiated cells were pre-incubated for 4 hours at 37° C. in Krebs-Ringer bicarbonate HEPES (KRBH) buffer of the following composition: 129 mM NaCl, 5 mM NaHCO3, 4.8 mM KCl, 1.2 mM KH2PO4, 1.2 mM MgSO4, 2.5 mM CaCl2, 10 mM HEPES and 0.1% (wt/vol) BSA at pH 7.4. For high glucose induced insulin release, cells were incubated in KRBH buffer supplemented with different concentration of glucose (3.3 mM and 16.7 mM) together with 10 μM tolbutamide (Sigma-Aldrich, St. Louis, Mo.) for 2 hours at 37° C. The concentration of insulin secreted into the culture media was measured using a human insulin enzyme-linked immunosorbent assay (ELISA) kit (CUSABIO, China). DNA of the cells were isolated by Trizol and determined using NanoDrop® 1000 spectrophotometer (NanoDrop products, Wilmington, Del.).

q-PCR analysis: as above.

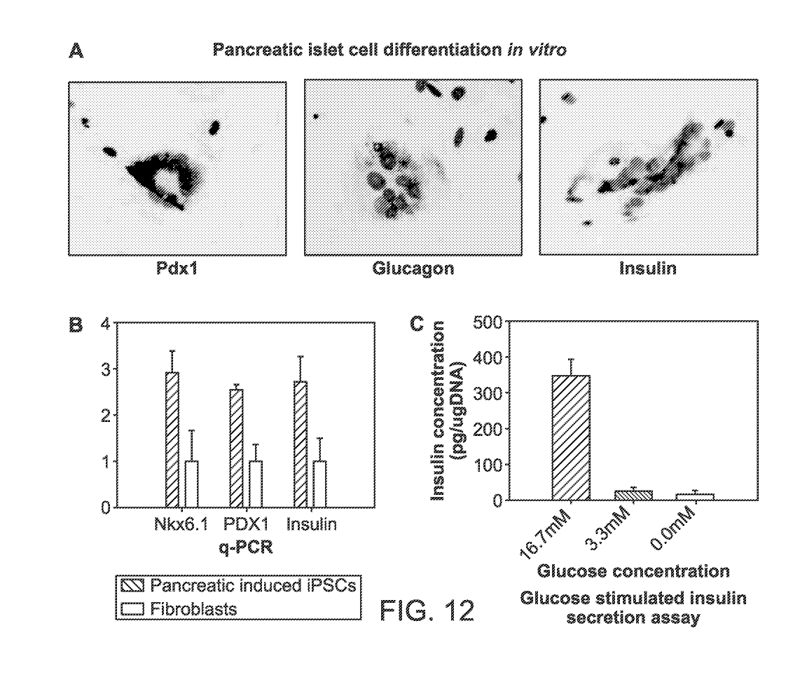

The results showed that iPSCs could be induced into islet cells over a 12 day period. After induction the fibroblasts formed typical endocrine aggregates and expressed pancreatic specific markers such as Pdx1, Glucagon and Insulin (FIG. 12A). The gene expression of Pdx1, Glucagon and Insulin in induced cells increased significantly compared with control group (FIG. 12B). After been exposure to high concentration glucose (16.7 mM), insulin released by inducted islet cells increased significantly compared with low concentration glucose (3.3 mM) and KRBH buffer without glucose (FIG. 12C).

### Example 5

### Identification of iPSCs Pluripotent Potential In Vivo

Osteogenic Differentiation and Identification of iPSCs In Vivo

Reprogrammed fibroblasts were seeded on a porous β-TCP scaffold and cultured in the osteogenic induction medium for three weeks. β-TCP showed high biocompatibility for induced fibroblasts, the cells adherented and proliferated on the porous β-TCP scaffold. After three weeks, the osteogenic fibroblastsβ-TCP scaffold constructs were implanted subcutaneously on the back of nude mice. Two months after implantation animals were sacrificed and the scaffolds were harvested and a histological analysis was performed as follows.

Osteogenic fibroblasts β-TCP scaffold constructs were fixed in freshly prepared 4% paraformaldehyde in PBS. Following fixation, the scaffolds were embedded in paraffin. Ten micron tissue sections were cut, prepared for histological analysis and stained with Harris Hematoxylin and Eosin (HE). Tissue sections were also stained by a modified Masson Trichrome Staining to demonstrate collagen synthesis. Von Kussa staining show the presence of matrix mineralization. HLA is simply the major histocompatibility complex (MHC) specific to humans. HLA-ABC immunohistochemistry staining confirmed the osteogenic tissue were originated from human cells.

HE staining was performed using standard methods (see for example IHC World).

Masson Trichrome Staining was performed using standard methods (see for example, Li, et al. Oral Surg Oral Med Oral Pathol Oral Radiol., 118(3):330-7 (2014)) and Von kussa staining was performed using standard methods (see for example, Subbiah, et al. Biomed Mater., 9(6):1-12 (2014)).

For immunohistochemistry staining of HLA-ABC, tissue sections were deparaffinized and rehydrated through 100% alcohol, 95% alcohol 70% alcohol washes, followed by washing in distilled water. Non-specific binding was blocked by incubating with 2% BSA/PBS (w/v) 37° C. for 60 minutes. Sections were then incubated with Ms mAb to HLA class I ABC (Abcam, Cabridge, Mass.) 1:100 4° C. overnight in a humid chamber. Sections were washed in PBS buffer and incubated with Anti-mouse HRP (Cell Signaling Technology, Danvers, Mass.) 1:1000 37° C. for 60 minutes, followed by washing in PBS buffer. DAB substrate solution was added to each slide and counterstained with hematoxylin.

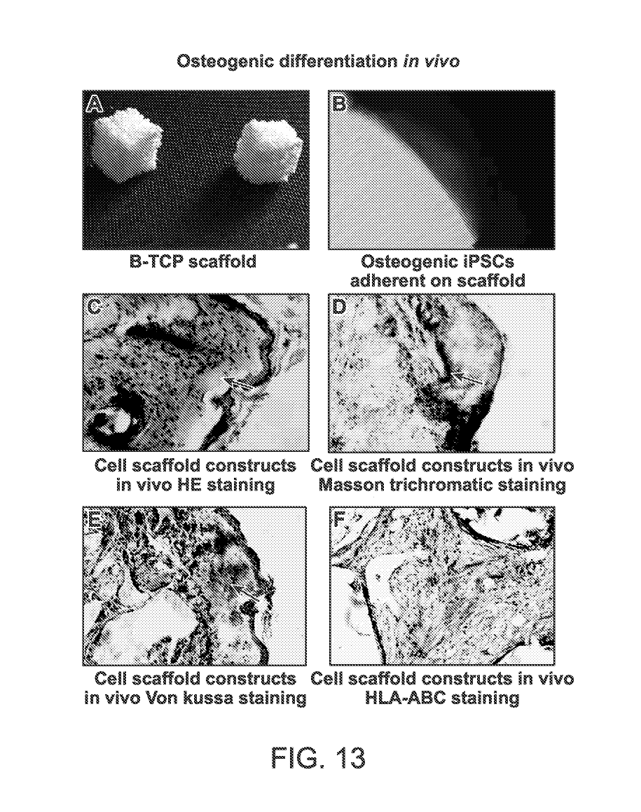

The results showed that differentiated iPSCs effectively adhered to and multipled on the β-TCP scaffold (FIG. 13A-B). Osteogenic fibroblasts β-TCP scaffold constructs were implanted subcutaneously on the back of nude mice. Two months after implantation animals were sacrificed and the scaffolds were harvested. HE staining show the structure of osteogenic tissue formed in vivo (FIG. 13C). Masson Trichrome staining shows the collagen fibers in the tissue (FIG. 13D). Von Kussa staining confirms the presence of matrix mineralization (FIG. 13E). HLA is simply the major histocompatibility complex (MHC) specific to humans. HLA-ABC immunohistochemistry staining confirmed the osteogenic tissue originated from human cells (FIG. 13F).

Adipogenic Differentiation and Identification of iPSCs In Vivo

Reprogrammed fibroblasts were seeded on a polyglycolic acids (PGA) scaffold and cultured in the adipogenic induction medium for three weeks. Induced fibroblasts were adherented and proliferated on the biocompatible porous PGA scaffold very well. After three weeks, the adipogenic fibroblasts PGA scaffold constructs were implanted subcutaneously on the back of nude mice. Two months after implantation animals were sacrificed and the scaffolds were harvested and a histological analysis was performed as described above for osteogenic fibroblasts.

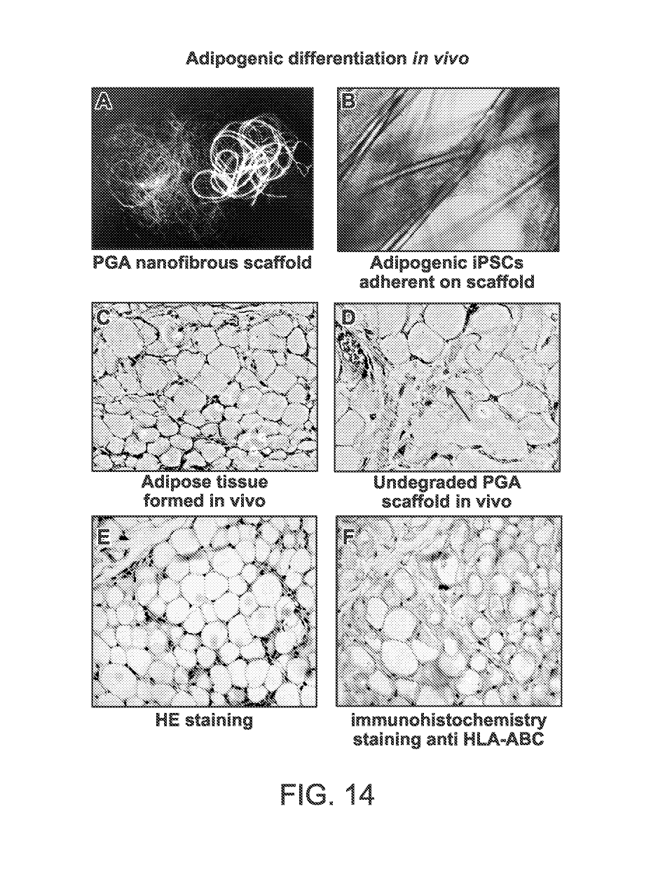

The results are shown in FIG. 14A-F using the scaffold for in vivo adipogenic differentiation (FIG. 14A). Reprogrammed fibroblasts were seeded on a polyglycolic acids (PGA) scaffold and cultured in the adipogenic induction medium for three weeks. Induced fibroblasts effectively adhered to and proliferated on the porous PGA scaffold (FIG. 14B). Adipogenic fibroblasts scaffold constructs were implanted subcutaneously on the back of nude mice for two months, at which stage cell scaffold constructs were found to have grown into adipose tissues in vivo. HE staining showed the adipogenic tissue structure that was formed in vivo (FIG. 14C, 14E): lipid vacuoles were observed. The arrow shows the undegraded PGA scaffold in vivo (FIG. 14D). HLA-ABC immunohistochemistry staining confirmed the adipogenic tissue originated from human cells (FIG. 14F).

### Example 6

### Teratomas Formation In Vivo

Reprogrammed fibroblast cell suspensions (1×10⁷) were mixed with matrigel and injected subcutaneously into SCID mice without anesthesia. After two months teratomas were collected and fixed in paraformaldehyde prior to HE staining and immunohistochemistry staining of HLA-ABC as described above.

The pluripotency of reprogrammed fibroblasts using HLA-ABC immunohistochemistry staining confirmed the teratoma originated from human cells.

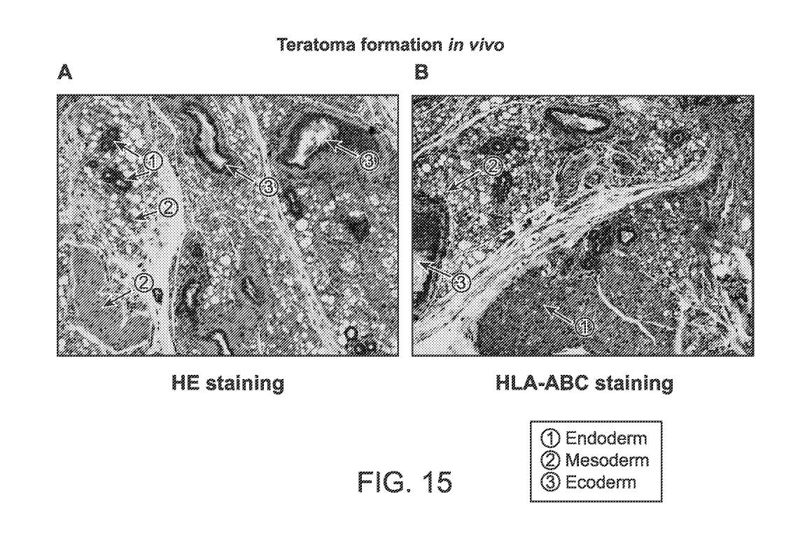

The results of reprogramming are shown in FIG. 15A-B. Reprogrammed fibroblasts suspension (1×10⁷) were mixed with matrigel and injected subcutaneously into SCID mice without anesthesia. After two months tissues from endoderm, mesoderm and ectoderm were formed in the teratoma (shown by HE staining), confirming the pluripotency of the reprogrammed fibroblasts (FIG. 15A). HLA-ABC immunohistochemistry staining confirmed the teratoma originated from human cells (FIG. 15B).

### Example 7

### Direct Adipogenic Reprogramming

Human foreskin fibroblast cells were transduced with transcription factors using HVJ-E 2 proteins: Oct4 8×10-3 μg/ml and C/EBPβ 1×10-3 μg/ml (Abnova, Taipei, Taiwan) made according to the description above were packaged into freeze-dried HVJ-envelopes using GenomONE-NeoEX HVJ Envelope Vector KIT using the protocol provided by the manufacturer.

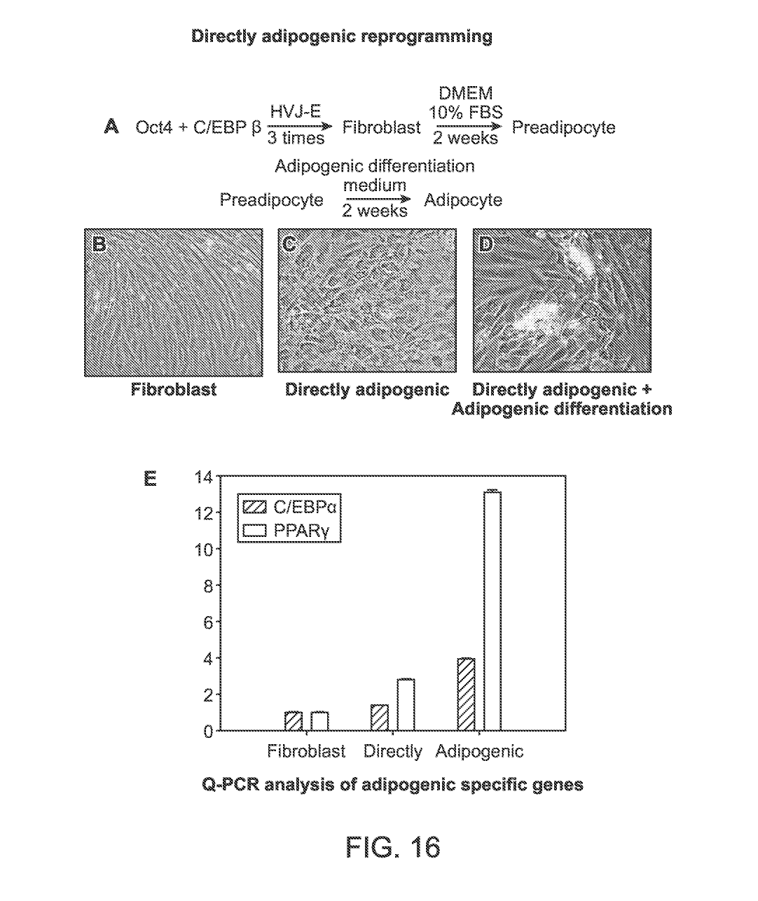

A summary of the timeline for fibroblast reprogramming is shown in FIG. 16A. On day 1, Oct4 and C/EBPβ were transduced into fibroblasts by HVJ-E, and a day later, the medium was replaced and cells cultured. A second transduction using the HVJ-E occurred on Day 4 and a third transduction occurred on day 7. On day 10, the transduced cells were plated on 6-well culture plate (Thermo Scientific, Waltham, Mass.) with 1×10⁵ cells per well and incubated at 37° C., 5% CO₂. The medium was changed to DMEM 10% FBS culture medium (Life Technologies, Carlsbad, Calif.). From Day 11 to 24, the cells were fed and monitored and the culture medium changed every other day. On day 25, the medium was changed to adipogenic differentiation medium described above. From Day 25 to 39, the reprogrammed pre-adipocyte were differentiated in adipogenic differentiation medium.

Q-PCR analysis was performed as described above.

The results are shown in FIG. 16A-E. Two weeks after directly adipogenic reprogramming (FIG. 16A) the fibroblasts (FIG. 16B) changed to round preadipocyte (FIG. 16C), then two weeks after induction in adipogenic differention media, lipid droplets appeared in the cells (FIG. 16D). Q-PCR transcript analysis confirmed expression of CCAAT Enhancer Binding Protein (c/EBPa) and PPARy (which are two adipogenic specific genes) increased gradually during the two step adipogenic differentiation (FIG. 16E). β-actin was used as a control for Q-PCR.

### Example 8

### Direct Neurogenic Reprogramming

Human foreskin fibroblast cells were transduced with transcription factors using HVJ-E 3 proteins: Sox2 8×10-3 μg/ml, GATA3 2×10-3 μg/ml (Abnova, Taipei, Taiwan) and NeuroD1 2×10-3 μg/ml (Abnova, Taipei, Taiwan) made according to the description above were packaged into freeze-dried HVJ-envelopes using GenomONE-NeoEX HVJ Envelope Vector KIT using the protocol provided by the manufacturer.

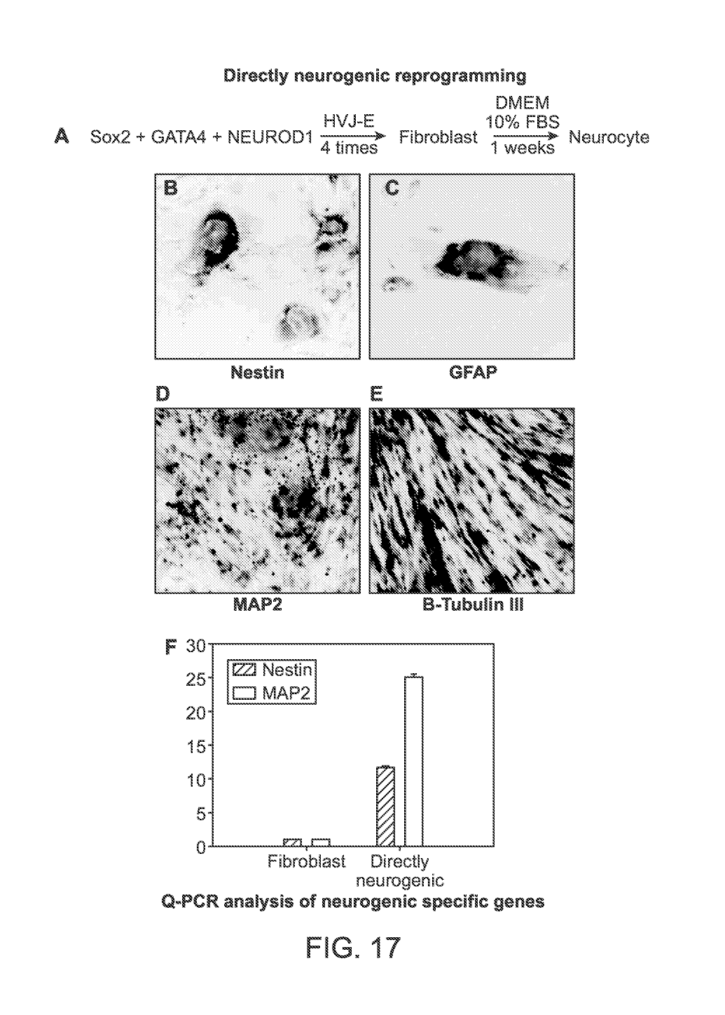

A summary of the timeline for fibroblast reprogramming is shown in FIG. 17A. On day 1, Sox2, GATA3 and NeuroD1 were transduced into fibroblasts by HVJ-E, and a day later, the medium was replaced and cells cultured. A second transduction using the HVJ-E occurred on Day 4, a third transduction occurred on day 7 and a fourth transduction occurred on day 10. On day 13, the transduced cells were plated on 6-well culture plate with 1×10⁵ cells per well cultured in DMEM 10% FBS culture medium (Life Technologies, Carlsbad, Calif.) incubated at 37° C., 5% CO₂. From Day 14 to 19, the cells were fed and monitored and the culture medium changed every other day.

Immunofluorescence staining Immunofluorescence staining by Anti-GFAP antibody 1:300, Anti-Nestin antibody 1:300, Anti-MAP2 antibody 1:200 and Anti-β-Tublin III 1:200 using a protocol above.

Q-PCR analysis was performed as described above.

The results are shown in FIG. 17A-F. The differentiated cells were positive for Nestin (FIG. 17B), GFAP (FIG. 17C), MAP2 (FIG. 17 D) and β-Tubullin III (FIG. 17E), which were all neuron-specific. The expression level of Nesin and MAP2 increased significantly after differentiation into neurogenic cells (FIG. 17F). β-actin was used as a control for the Q-PCR.

### Example 9

### Effect of Different Transcription Factors on Reprogramming

Human foreskin fibroblast cells were transduced with different combination of transcription factors using HVJ-E. The protocol of transcription factors transduction is described above. Flow cytometry analysis, Alkaline Phosphatase (AKP) activity assay, Oil Red 0 Staining; RT-PCR and determination of adipogenic and osteogenic differentiation and identification of iPSCs in vitro were performed as described above.

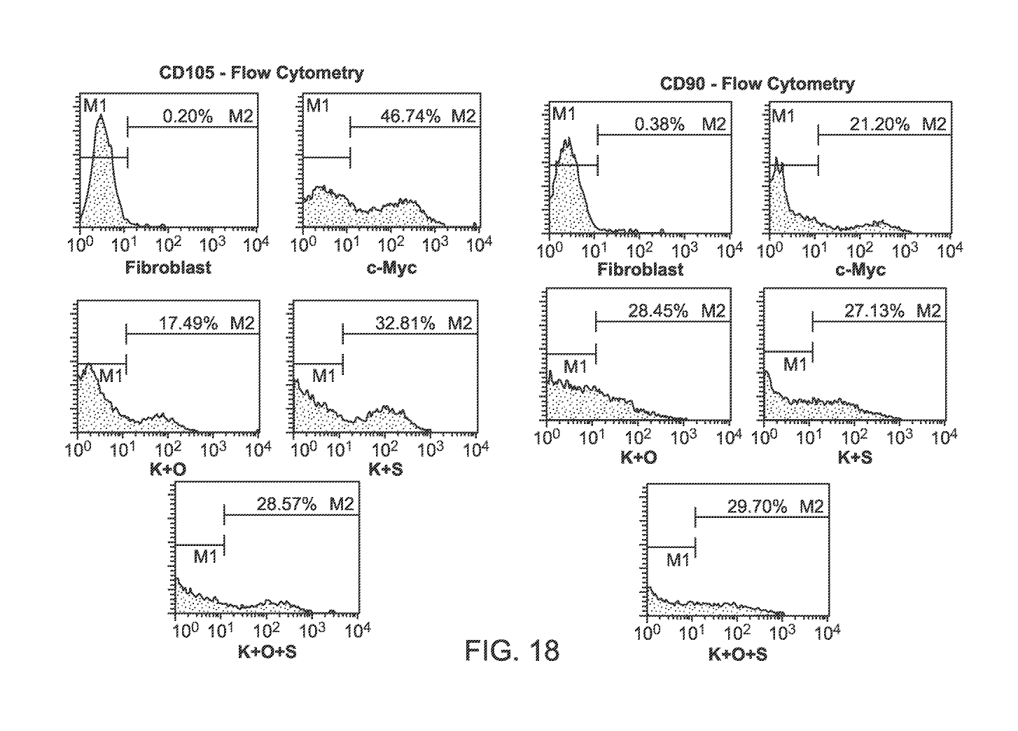

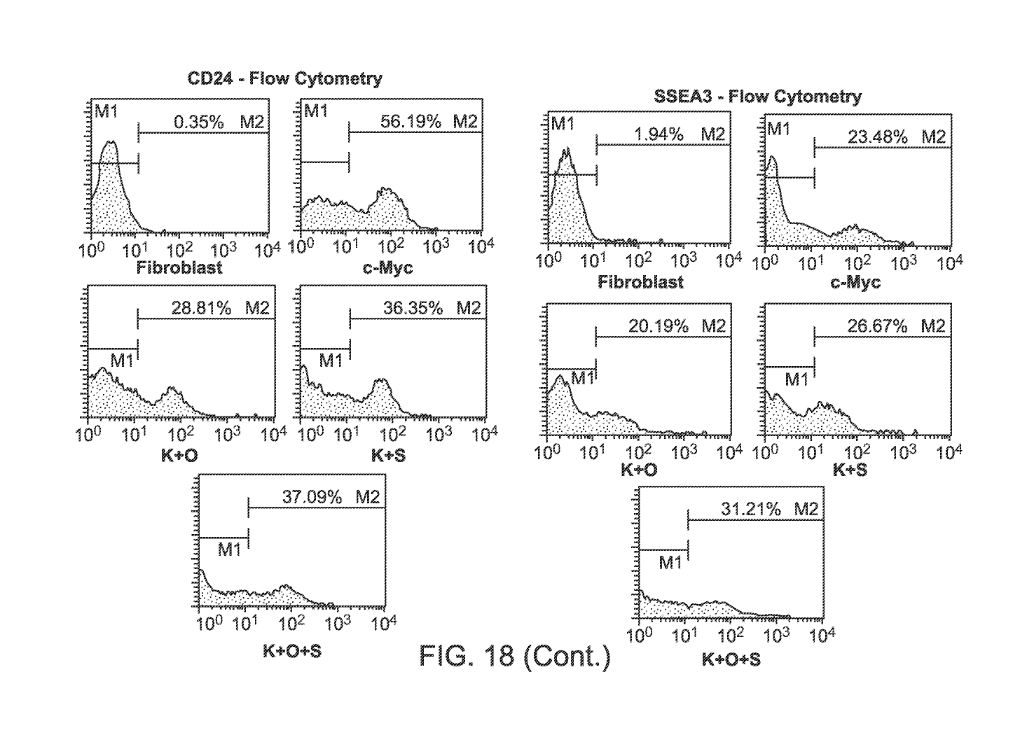

The results are shown in FIGS. 18 to 21.

FIG. 18 shows results of FACS analysis of fibroblasts that were transfected with one, two or three of the transcription factors O, S, K and c-Myc. After transfection with different combinations of the transcription the factors (O, S, K and c-Myc), stem cell surface markers all increased significantly. This indicates the different combination of transcription factors can reprogram fibroblasts to some extent. Except CD105, stem cell surface marker expression was the highest in K+O+S group.

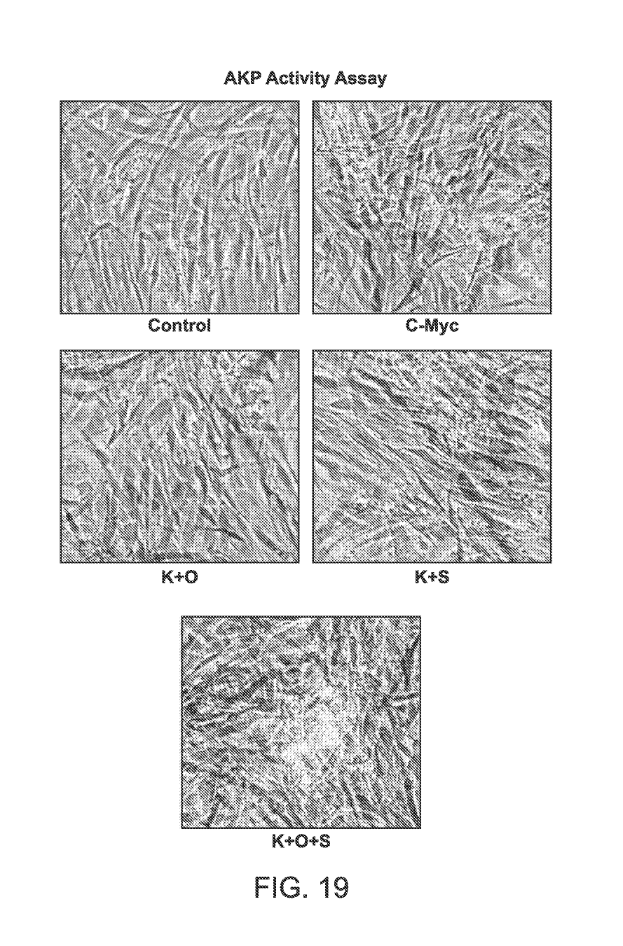

FIG. 19 shows the results of an AKP activity assay. After reprogramming by various transcription factors, AKP activity increased significantly. This indicates the different combination of transcription factors all have reprogramming abilities. But the percentage of AKP positive cells in each group was different, K+O+S group showed the highest percentage. The reprogramming efficiency increased according to the transcription factors added.

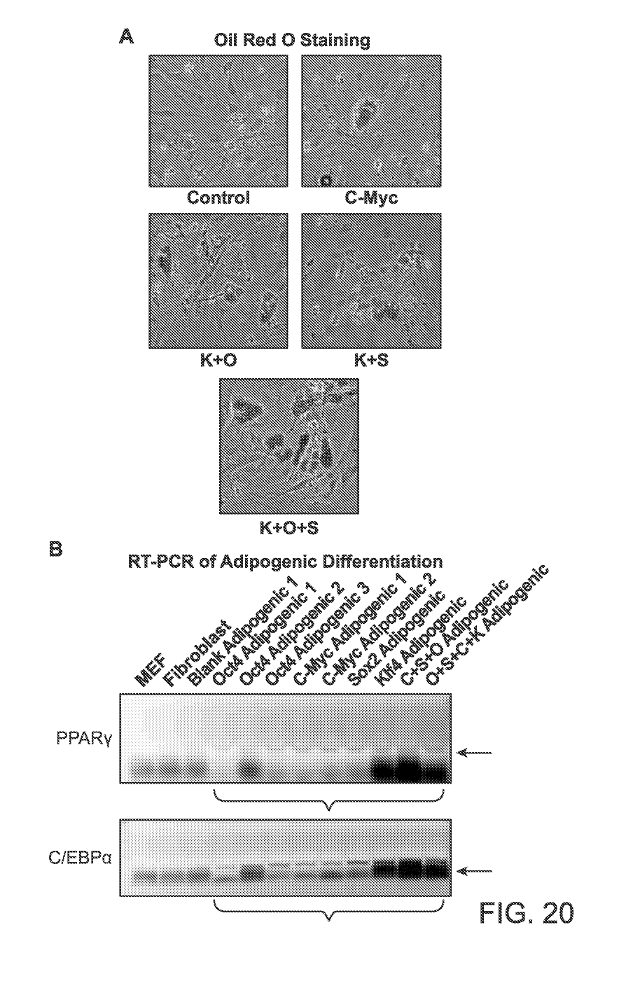

FIG. 20A-B shows that cells transduced with combinations of the four transcription factors (O, S, K and c-Myc) can differentiate into adipocytes. Reprogrammed stem cells were induced into adipocytes by the medium with 1 μM Dexamethasone, 200 μM Indomethacin, 500 μM IBMX and 10 μg/ml Insulin. Cells could be induced into adipocytes as indicated by Oil Red staining (FIG. 20A). But the percentage of adipocyte in each group was different, K+O+S group showed the highest percentage. The induced efficiency increased according to which transcription factors added. The expression of PPARy and C/EBPa were induced in different adipogenic group (FIG. 20B).

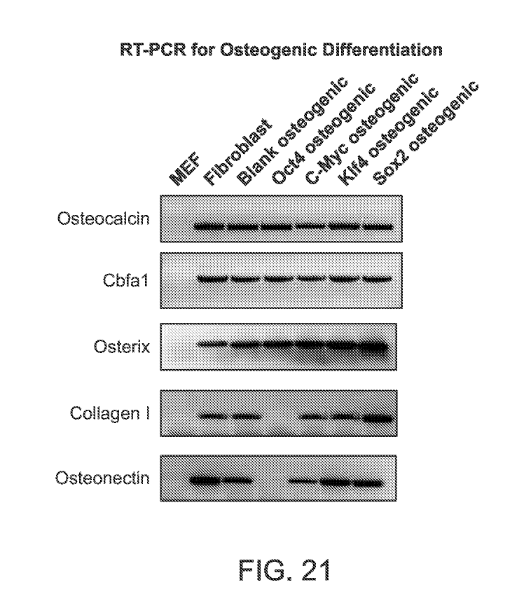

FIG. 21 shows that cells transduced with combinations of the four transcription factors can differentiate into osteoblasts although some of the osteoblast-specific genes expressed in different osteogenic groups were also expressed in iPSCs.

The data shown above demonstrates that viral delivery of one or more isolated transcription factor proteins selected from the group consisting of Sox2, Oct4, Klf4 and c-Myc, packaged within the particle, can result in highly efficient reprogramming of a) somatic cells to pluripotent stem cells and b) highly efficient reprogramming of somatic cells into differentiated cell types (e.g., adipocytes and neurons). Some of these results are summarized in the following table.

TABLE 1
Primers for RT-PCT Reactions

| Gene Name | Forward Primer | Reverse Primer |
|---|---|---|
| PPARγ | TTCAGCAGCGTGTTCGACTT (SEQ ID NO: 11) | AGGAATCGCTTTCTGGGTCA (SEQ ID NO: 12) |
| C/EBPα | CTAACTCCCCCATGGAGTCGG (SEQ ID NO: 13) | GTCGATGGACGTCTCGTGC (SEQ ID NO: 14) |
| Collagen I | GATGGATTCCAGTTCGAGTATG (SEQ ID NO: 15) | GTTTGGGTTGCTTGTCTGTTTG (SEQ ID NO: 16) |
| Cbfa I | GATGACACTGCCACCTCTGA (SEQ ID NO: 17) | GACTGGCGGGGTGTAAGTAA (SEQ ID NO: 18) |
| Osteocalcin | ATGAGAGCCCTCACACTCCTC (SEQ ID NO: 19) | CGTAGAAGCGCCGATAGGC (SEQ ID NO: 20) |
| Osterix | TAATGGGCTCCTTTCACCTG (SEQ ID NO: 21) | CACTGGGCAGACAGTCAGAA (SEQ ID NO: 22) |
| Bone | TCAGCATTTTGGGAATGGCC (SEQ ID NO: 23) | GAGGTTGTTGTCTTCGAGGT (SEQ ID NO: 24) |
| Sialoprotein | | |
| Osteonectin | AGTAGGGCCTGGATCTTCTT (SEQ ID NO: 25) | CTGCTTCTCAGTCAGAAGGT (SEQ ID NO: 26) |
| Nestin | AGCTGGCGCACCTCAAGATG (SEQ ID NO: 27) | AGGGAAGTTGGGCTCAGGAC (SEQ ID NO: 28) |
| GFAP | GAGGCGGCCAGTTATCAGGA (SEQ ID NO: 29) | GTTCTCCTCGCCCTCTAGCA (SEQ ID NO: 30) |

TABLE 2
Primers for q-PCR reactions

| Gene Name | Forward Primer | Reverse Primer |
|---|---|---|
| β-actin | CATGTACGTTGCTATCCAGGC (SEQ ID NO: 31) | CTCCTTAATGTCACGCACGAT (SEQ ID NO: 32) |
| Klf4 | CCCACATGAAGCGACTTCCC (SEQ ID NO: 33) | CAGGTCCAGGAGATCGTTGAA (SEQ ID NO: 34) |
| Nanog | TTTGTGGGCCTGAAGAAAACT (SEQ ID NO: 35) | AGGGCTGTCCTGAATAAGCAG (SEQ ID NO: 36) |
| c-Myc | GGCTCCTGGCAAAAGGTCA (SEQ ID NO: 37) | CTGCGTAGTTGTGCTGATGT (SEQ ID NO: 38) |
| Sox2 | GCCGAGTGGAAACTTTTGTCG (SEQ ID NO: 39) | GGCAGCGTGTACTTATCCTTCT (SEQ ID NO: 40) |
| hTERT | TAATGGGCTCCTTTC ACCTG (SEQ ID NO: 41) | CAGTGCGTCTTGAGGAGCA (SEQ ID NO: 42) |
| ABCG2 | CAGGTGGAGGCAAATCTTCGT (SEQ ID NO: 43) | ACCCTGTTAATCCGTTCGTTTT (SEQ ID NO: 44) |
| REX1 | GCAGCCACGGCCTATTAAG (SEQ ID NO: 45) | CCACCACGTACTTGCCACT (SEQ ID NO: 46) |
| Insulin | GCAGCCTTTGTGAACCAACAC (SEQ ID NO: 47) | CCCCGCACACTAGGTAGAGA (SEQ ID NO: 48) |
| Pdx1 | ATCTCCCCATACGAAGTGCC (SEQ ID NO: 49) | CGTGAGCTTTGGTGGATTTCAT (SEQ ID NO: 50) |
| Nkx6.1 | GGACTGCCACGCTTTAGCA (SEQ ID NO: 51) | TGGGTCTCGTGTGTTTTCTCT (SEQ ID NO: 52) |
| C/EBPα | CTTCAGCCCGTACCTGGAG (SEQ ID NO: 53) | GGAGAGGAAGTCGTGGTGC (SEQ ID NO: 54) |
| PPARγ | GGGATCAGCTCCGTGGATCT (SEQ ID NO: 55) | TGCACTTTGGTACTCTTGAAGTT (SEQ ID NO: 56) |

TABLE 3
Transcription Factors

| | | | | Directly | | | | |
|---|---|---|---|---|---|---|---|---|
| | | | | pancreatic | Directly | Directly | Directly | Directly |
| | | Directly | Directly | islet | hepatic | myogenic | cardiomyocyte | osteogenic |
| | iPSCs | neurogenic | adipogenic | differentiation | differentiation | differentiation | differentiation | differentiation |
| c-Myc, | X | | | | | | | |
| Nanog | | | | | | | | |
| SOX2 | X | X | | | | | | |
| KLF4 | X | | | | | | | |
| OCT3/4 | X | | | | | | | |
| LIN28 | X | | | | | | | |
| TERT | X | | | | | | | |
| BM2, | | X | | | | | | |
| MYT11, | | | | | | | | |
| Asc1 | | | | | | | | |
| NeuroD 1 | | X | | X | | | | |
| NeuroD 2 | | X | | | | | | |
| Gata4 | | X | | | X | | X | |
| Pou3f2 | | X | | | | | | |
| PPARγ, | | | X | | | | | |
| C/EBPα, | | | | | | | | |
| C/EBPβ | | | | | | | | |
| SREBP-1 | | | X | | | | | |
| Pdx1 | | | | X | | | | |
| MafA | | | | X | | | | |
| Ngn3 | | | | X | | | | |
| Nkx6.1 | | | | X | | | | |
| Nkx2.2 | | | | X | | | | |
| FOXa2 | | | | X | | | | |
| Mafb | | | | X | | | | |
| Hnf1 α | | | | | X | | | |
| Hnf4 α | | | | | X | | | |
| Foxa 1 | | | | | X | | | |
| Foxa2 | | | | | X | | | |
| Foxa3 | | | | | X | | | |
| MyoD | | | | | | X | | |
| Myf4 | | | | | | X | | |
| MRF4 | | | | | | X | | |
| Mef2c | | | | | | | X | |
| Tbx5 | | | | | | | X | |
| Nkx2-5 | | | | | | | X | |
| RhoA | | | | | | | X | |
| BMP2 | | | | | | | X | |
| BMP4 | | | | | | | | X |
| Runx2 | | | | | | | | X |
| Vdr | | | | | | | | X |
| Opn | | | | | | | | X |
| Osf2 | | | | | | | | X |

TABLE 4
Transcription Factors Concentration

| | Transcription factors concentration | | | |
|---|---|---|---|---|
| iPSCs | Sox2 8 × | Oct4 8 × | Klf4 8 × | c-Myc 8 × |
| reprogramming | 10−3 μg/ml | 10−3 μg/ml | 10−3 μg/ml | 10−3 μg/ml |
| Directly | Oct4 8 × | C/EBPβ 1 × | | |
| adipogenic | 10−3 μg/ml | 10−3 μg/ml | | |
| reprogramming | | | | |
| Directly | Sox2 8 × | GATA3 2 × | NeuroD1 3 × | |
| neurogenic | 10−3 μg/ml | 10−3 μg/ml | 10−3 μg/ml | |
| reprogramming | | | | |

What is claimed is:

1. An inactivated viral particle comprising:
(a) an envelope; and
(b) one or more isolated transcription factor proteins.

2. An inactivated viral particle wherein the one or more transcription factor proteins are selected from the group consisting of Sox2, Oct4, Klf4, c-Myc, C/EBPβ, GATA3 and NeuroD1, packaged within the particle.

3. The inactivated viral particle of claim 1, wherein the inactivated viral particle is an inactivated Sendai virus, herpesvirus, parainfluenza virus or lentivirus particle comprising an HVJ envelope and one or more of the isolated transcription factor proteins packaged within the particle.

4. The inactivated viral particle of claim 1, wherein the inactivated viral particle is an inactivated Sendai viral particle comprising an HVJ envelope.

5. The inactivated viral particle of claim 1, wherein the particle comprises:
(a) a Sox2 transcription factor, wherein the Sox2 transcription factor has an amino acid sequence that is at least 80% identical to a mammalian Sox2 protein;
(b) an Oct4 transcription factor, wherein the Oct4transcription factor has an amino acid sequence that is at least 80% identical to mammalian Oct4 protein;
(c) a Klf4 transcription factor, wherein the Klf4 transcription factor has an amino acid sequence that is at least 80% identical to mammalian Klf4 protein; and
(d) a c-Myc transcription factor, wherein the c-Myc transcription factor has an amino acid sequence that is at least 80% identical to mammalian c-Myc protein; packaged within the particle.

6. The inactivated viral particle according to claim 1, wherein the particle comprises:
(a) an Oct4 transcription factor, wherein the Oct4 transcription factor has an amino acid sequence that is at least 80% identical to a mammalian Oct4 protein; and
(b) an C/FEPβ transcription factor, wherein the C/EBPβ transcription factor has an amino acid sequence that is at least 80% identical to mammalian C/EBPβ protein; packaged within the particle.

7. The inactivated viral particle of claim 1, wherein the particle comprises:
(a) an Sox2 transcription factor, wherein the Sox2 transcription factor has an amino acid sequence that is at least 80% identical to a mammalian Sox2 protein;
(b) an GATA3 transcription factor, wherein the GATA3 transcription factor has an amino acid sequence that is at least 80% identical to mammalian GATA3 protein;
(c) a NeuroD1 transcription factor, wherein the NeuroD1 transcription factor has an amino acid sequence that is at least 80% identical to mammalian NeuroD1 protein;
packaged within the particle.

8. A method comprising:
transfecting somatic cells with an inactivated viral particle according to claim 1, thereby introducing the one or more transcription factor proteins into the somatic cells and causing the somatic cells to develop into a reprogrammed cell type.

9. The method of claim 8, wherein the inactivated viral particle comprises one or more of Sox2, Oct4, Klf4 and c-Myc and the method causes the somatic cells to develop into pluripotent stem cells.

10. The method of claim 8, wherein the inactivated viral particle comprises Sox2, Oct4, Klf4 and c-Myc and the method causes the somatic cells to develop into pluripotent stem cells.

11. The method of claim 8, wherein the inactivated viral particle comprises Oct4 and C/EBPβ and the method causes the somatic cells to develop into adipocytes.

12. The method of claim 8, wherein the inactivated viral particle comprises isolated Sox2, GATA3 and NeuroD1 proteins and the method causes the somatic cells to develop into neurons.

13. The method of claim 8, wherein the transfecting comprises administering the inactivated viral particle to an animal.

14. The method according to claim 8, wherein the transfecting is done in vitro, and the method comprises culturing the somatic cells on a growth medium to produce the different reprogrammed cell type.

15. The method according to claim 8, wherein the somatic cells are fibroblasts.

16. The method according to claim 8, wherein the different cell type is a pluripotent stem cell.

17. The method of claim 16, further comprising: culturing the induced pluripotent stem cells on a differentiation medium to cause the induced pluripotent stem cell to differentiate into a differentiated cell type.

18. The method of claim 17, further comprising:
introducing the differentiated cells into a recipient subject in need of the differentiated cells.

19. The method of claim 17, further comprising:
seeding the induced pluripotent stem cells on a decellularized scaffold for an organ or tissue; and
causing the IPSCs to differentiate on the scaffold, thereby producing an artificial organ or tissue.

20. The method of claim 20, further comprising transplanting the recellularized organ or tissue into a recipient subject.

21. A method of making an inactivated viral particle according to claim 1, comprising: combining one or more transcription factor proteins with HVJ envelope in the presence of a detergent.

22. The method of claim 21, further comprising centrifuging the one or more transcription factor proteins, HVJ envelope and detergent to collect an inactivated viral particle comprising the one or more transcription factor proteins packaged therein.

23. A screening method comprising:
(a) transfecting somatic cells with an inactivated viral particle wherein the inactivated virus particle comprises:
(i) an envelope; and one or more isolated transcription factor proteins, or
(ii) an envelope and one or more transcription factor proteins selected from the group consisting of Sox2, Oct4, Klf4, c-Myc, C/EBPβ, GATA3 and NeuroD1, packaged within the particle; or
(iii) an inactivated Sendai virus, herpesvirus, parainfluenza virus or lentivirus particle comprising an HVJ envelope and one or more of the isolated transcription factor proteins packaged within the particle; or
(iv) an inactivated Sendai viral particle comprising an HVJ envelope; or
(v) a Sox2 transcription factor having an amino acid sequence that is at least 80% identical to a mammalian Sox2 protein; an Oct4 transcription factor, having an amino acid sequence that is at least 80% identical to mammalian Oct4 protein; a Klf4 transcription factor, having an amino acid sequence that is at least 80% identical to mammalian Klf4 protein; and c-Myc transcription factor, having an amino acid sequence that is at least 80% identical to mammalian c-Myc protein; packaged within the particle; or
(vi) an Oct4 transcription factor, having an amino acid sequence that is at least 80% identical to a mammalian Oct4 protein; and an C/EBPβ transcription factor, having an amino acid sequence that is at least 80% identical to mammalian C/EBPβ protein; packaged within the particle; or
(vii) a Sox2 transcription factor having an amino acid sequence that is at least 80% identical to a mammalian Sox2 protein; an GATA3 transcription factor having an amino acid sequence that is at least 80% identical to mammalian GATA3 protein; and
(b) contacting a test agent with the somatic cells;
(c) culturing the somatic cells; and
(d) determining whether the test agent has any effect on the cell type produced by culturing step (c).

24. The method of claim 23 wherein:
the culturing step (c) comprises culturing the somatic cells on pluripotent stem cell induction medium; and
the determining step (d) comprises determining whether the test agent has any effect on the induction of pluripotent stem cells.

25. The method of claim 23, wherein:
the culturing step (c) comprises culturing the somatic cells on pluripotent stem cell induction medium to produce pluripotent stem cells and, optionally, culturing the pluripotent stem cells on a differentiation medium; and
the determining step (d) comprises determining whether the test agent has any effect on the differentiation of a second type of somatic cells grown on the differentiation medium, wherein the second type of somatic cells is different to the somatic cells of step (b).

26. The method of claim 23, wherein the test agent is a small molecule.

27. The method of claim 23, wherein the test agent is a protein.

28. The method of claim 27, wherein the protein is packaged within the inactivated viral particle.

29. A screening method comprising:
(a) packaging a test agent within an inactivated viral particle in the absence of isolated transcription factor proteins or nucleic acid encoding the same;
(b) transfecting an induced pluripotent stem cell with the inactivated viral particle of step (a);
(c) culturing the transfected cells on a differentiation medium; and
(d) determining whether the test agent has any effect on the cell type produced by culturing step.

30. The method according to claim 8, comprising analyzing reprogramming by QPCR analysis.

31. The method according to claim 8, comprising analyzing reprogramming by cell morphology using cell stains.

32. The method according to claim 8, comprising analyzing reprogramming by analysis of metabolites characteristic of the reprogrammed cells
